SEQUENCE LISTING (25 sequences)

SEQ ID NO: 1 - RNA, 77 nt, Mus musculus
gcuugggacacauacuucuuuauaugcccauaugaaccugcuaagcuauggaauguaaagaaguauguauuucaggc

SEQ ID NO: 2 - RNA, 22 nt, Mus musculus
uggaauguaaagaaguauguau

SEQ ID NO: 3 - RNA, 68 nt, Mus musculus
gcuaaagcugguaaaauggaaccaaaucgccucuucaauggauuugguccccuucaaccagcuguagc

SEQ ID NO: 4 - RNA, 22 nt, Mus musculus
uuugguccccuucaaccagcug

SEQ ID NO: 5 - RNA, 73 nt, Mus musculus
ccaggccacaugcuucuuuauauccucauagauaucucagcacuauggaauguaaggaagugugugguuuugg

SEQ ID NO: 6 - RNA, 22 nt, Mus musculus
uggaauguaaggaagugugugg

SEQ ID NO: 7 - RNA, 83 nt, Mus musculus
uuccuuugacgggugagcuuuuggcccggguuauaccugacacucacguauaagacgagcaaaaagcuuguuggucagaggag

SEQ ID NO: 8 - RNA, 22 nt, Mus musculus
auaagacgagcaaaaagcuugu

SEQ ID NO: 9 - RNA, 71 nt, Homo sapiens
ugggaaacauacuucuuuauaugcccauauggaccugcuaagcuauggaauguaaagaaguauguaucuca

SEQ ID NO: 10 - RNA, 85 nt, Homo sapiens
accuacucagaguacauacuucuuuauguacccauaugaacauacaaugcuauggaauguaaagaaguauguauuuuugguaggc

SEQ ID NO: 11 - RNA, 22 nt, Homo sapiens
uggaauguaaagaaguauguau

SEQ ID NO: 12 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 13 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 14 - RNA, 22 nt, Homo sapiens
uuugguccccuucaaccagcug

SEQ ID NO: 15 - RNA, 86 nt, Homo sapiens
ugcuucccgaggccacaugcuucuuuauauccccauauggauuacuuugcuauggaauguaaggaagugugugguuucggcaagug

SEQ ID NO: 16 - RNA, 22 nt, Homo sapiens
uggaauguaaggaagugugugg

SEQ ID NO: 17 - RNA, 71 nt, Homo sapiens
ugacgggcgagcuuuuggcccggguuauaccugaugcucacguauaagacgagcaaaaagcuuguugguca

SEQ ID NO: 18 - RNA, 22 nt, Homo sapiens
auaagacgagcaaaaagcuugu

SEQ ID NO: 19 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 20 - RNA, 84 nt, Homo sapiens
cguugcugcagcugguguugugaaucaggccgacgagcagcgcauccucuuacccggcuauuucacgacaccaggguugcauca

SEQ ID NO: 21 - RNA, 23 nt, Homo sapiens
agcugguguugugaaucaggccg

SEQ ID NO: 22 - DNA, 1284 bp, Homo sapiens
cacacccacacgagatgggcacggagtagtcttgaaagacatgacaaatcaccagacctgggaagaagctaaagagccagagggaaaaagccagaagtcgactacctgggaggagggatagacaagaaaccaaactaaaggaaactaagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaa

SEQ ID NO: 23 - protein, 289 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 24 - RNA, 79 nt, Mus musculus
gggugggcagcuguuaagacuugcagugauguuuagcuccucugcaugugaacaucacagcaagucugugcugcugccu

SEQ ID NO: 25 - RNA, 21 nt, Mus musculus
uuaagacuugcagugauguuu

(19) United States
(12) Patent Application Publication
(10) Pub. No.: US 2020/0009197 A1
(43) Pub. Date: Jan. 9, 2020

(54) COMPOSITIONS AND METHODS FOR THE REPROGRAMMING OF CELLS INTO CARDIOMYOCYTES

(71) Applicant: Duke University, Durham, NC (US)

(72) Inventors: Xiaowen Wang, St. Louis, MO (US); Conrad P. Hodgkinson, Durham, NC (US); Victor Dzau, Durham, NC (US)

(21) Appl. No.: 16/447,672

(22) Filed: Jun. 20, 2019

Related U.S. Application Data
(63) Continuation of application No. 15/502,451, filed on Feb. 7, 2017, now abandoned.
(60) Provisional application No. 62/034,365, filed on Aug. 7, 2014.

(51) Int. Cl. A61K 35/34; A61K 35/12

(52) U.S. Cl. CPC A61K 35/34; A61K 35/12

(57) ABSTRACT

The present disclosure provides compositions and methods for the reprogramming of cells such as fibroblasts into cardiomyocytes. The invention provided herein features a chemically defined media and methods of reprogramming cells to increase cardiac gene and protein expression in cardiac fibroblasts and other fibroblasts, e.g. dermal fibroblasts. The media and methods also enhance miR-combo mediated cardiac reprogramming of fibroblasts to cardiomyocytes. Thus, the invention encompasses a chemically defined reprogramming media comprising a base tissue culture media, insulin-transferrin-selenium (ITS) or ascorbic acid in a somatic cell-reprogramming, e.g., fibroblast-to-cardiomyocyte-reprogramming, amount.

### CROSS REFERENCE TO RELATED APPLICATIONS

This application is a continuation of U.S. patent application Ser. No. 15/502,451, filed Feb. 7, 2017, which is a national stage application filed under 35 U.S.C. § 371, of International Application No.: PCT/US2015/044354, filed Aug. 7, 2015, and claims the benefit of U.S. Provisional Patent Application No. 62/034,365, filed Aug. 7, 2014, the contents of each of which are incorporated by reference herein in its entirety.

### GOVERNMENT INTEREST

This invention was made with Government support under Federal Grant No. R01-HL081744-07 awarded by the National Institutes of Health (NIH). The Government has certain rights to this invention.

### INCORPORATION BY REFERENCE OF SEQUENCE LISTING

The contents of the text file named “35327-515N01US_ST25.txt”, which was created on Jun. 28, 2017 and is 12,288 bytes in size, are hereby incorporated by reference in their entireties and for all purposes.

### FIELD OF THE INVENTION

This invention relates generally to the field of cardiology and repair of cardiac tissue following injury.

### BACKGROUND

Heart failure is a disease affecting over 5 million people in the U.S., with 825,000 new cases each year (see, e.g., Go, A. S. et al. (2014) Circulation 129:e28-e292). The heart has low intrinsic regenerative ability and excessive fibrosis (i.e. formation of scar tissue) following a myocardial ischemic event results in significant impairment of tissue structure and function (see, e.g., Burridge, P. W. et al. (2012) Cell Stem Cell 10:16-28). Cardiac reprogramming, whereby cardiomyocytes are regenerated from cells inhabiting the scar tissue or from other cells derived from a subject, is an exciting field with a high degree of interest due to its therapeutic potential. For example, several groups have used transcription factors and small molecules to reprogram cardiac fibroblasts into cardiomyocytes (see, e.g., Ieda, M. et al. (2010) Cell 142:375-386; Qian, L. et al. (2012) Nature 485:593-598; Wang, H. et al. (2014) Cell Reports 6:951-960). Research has also shown that the use of a combination of microRNAs (miR-combo) can induce reprogramming (see, e.g., Jayawardena, T. M. et al. (2012) Circ. Res. 110:1465-1473). However, in spite of this progress, reprogramming efficiency remains low thereby limiting therapeutic potential. Therefore, new compositions and methods are needed to enhance cardiac reprogramming of cells to cardiomyocytes for therapeutic application following injury to cardiac tissue.

### SUMMARY

The invention provided herein features a chemically defined media and methods of reprogramming cells to increase cardiac gene and protein expression in cardiac fibroblasts and other fibroblasts, e.g. dermal fibroblasts. The media and methods also enhance miR-combo mediated cardiac reprogramming of fibroblasts to cardiomyocytes. Thus, the invention encompasses a chemically defined reprogramming media comprising a base tissue culture media, insulin-transferrin-selenium (ITS) or ascorbic acid in a somatic cell-reprogramming, e.g., fibroblast-to-cardiomyocyte-reprogramming, amount. The media may further comprise bovine serum albumin (BSA) or L-glutamine A somatic cell reprogramming amount of insulin-transferrin-selenium is characterized by insulin being present in an amount of 10 nanomolar to 10 micromolar (e.g., 100 nM), transferrin being present in an amount of 0.002 to 1 gram per liter (e.g., 0.055 g/1), and selenium being present in an amount of 1-100 μg per liter (e.g., 6.7 μg per liter. Optionally, the media comprises 0.2 mM to 20 mM L-glutamine (e.g., 2 mM). The media may also optionally include 50 μM to 50 millimolar ascorbic acid such as 100-500 μM, e.g, 250 μM, of ascorbic acid.

The components of insulin:selenium:transferrin are present in the following ratios. Insulin range, 200 to 10000 with respect to selenium; transferrin range, 50 to 5000 with respect to selenium. Insulin to transferrin ratio is 1.2 to 12:1, e.g., 1.8:1. An exemplary ratio of insulin:selenium:transferrin is 1493:1:821, respectively.

Accordingly, in one aspect, provided herein is a chemically defined reprogramming media comprising a cell culture medium; e.g., advanced-Dulbecco's Modified Eagle Medium/Nutrient Mixture F12 media, purified bovine serum albumin (BSA), insulin-transferrin-selenium (ITS), L-glutamine, and ascorbic acid. In one embodiment, the media comprises 0.2% purified BSA. In some embodiments of any of the embodiments disclosed herein, the media comprises 1× insulin-transferrin-selenium. In some embodiments of any of the embodiments disclosed herein, the media comprises 1× L-glutamine. In one example the media comprises 250 μM ascorbic acid. In some examples the media further comprises a reprogramming efficiency-enhancing molecule. Exemplary molecules include one or more molecules selected from the group consisting of valproic acid, bone morphogenetic protein 4 (BMP4), JAK inhibitor 1, RG108, R(+)Bay K 8644, PS48, and A83-01. One embodiment of the present disclosure provides a chemically defined reprogramming media comprising, consisting of, or consisting essentially of advanced-DMEM/F12 media, 0.2% bovine serum albumin, 1× insulin-transferrin-selenium, 1× L-glutamine, and 250 μM ascorbic acid.

In another aspect, provided herein are methods for reprogramming a fibroblast cell e.g., cardiac fibroblast or skin (dermal) fibroblast, the method comprising contacting the cell with the media of any of the embodiments disclosed herein for a sufficient amount of time and volume such that the fibroblast is reprogrammed into a cardiomyocyte. In some embodiments, the method further comprises transfecting into the cell at least one microRNA (miRNA) that mediates direct reprogramming of cells into cardiomyocytes prior to culturing in the chemically defined reprogramming media. Exemplary microRNA oligonucleotides are described in U.S. Publication No. 20140011281 hereby incorporated by reference. In some embodiments, the miRNA is selected from the group consisting of miR-1, miR-133, miR-208, miR-499 and combinations thereof. The cell is selected from the group consisting of fibroblasts, adipocytes, or CD34+ umbilical cord blood cells. For example, the fibroblast is a cardiofibroblast or other fibroblast such as a dermal fibroblast. In some embodiments the cell comprises cardiac fibrotic tissue. The method further optionally comprises contacting the cell with a reprogramming efficiency-enhancing molecule such as a microRNA described above or more molecules selected from the group consisting of valproic acid, bone morphogenetic protein 4 (BMP4), JAK inhibitor 1, RG108, R(+)Bay K 8644, PS48, and A83-01.

Another aspect of the present disclosure provides a method of reprogramming a fibroblast (e.g., cardiac or dermal fibroblast) into a cardiomyocyte in a subject in need thereof comprising, consisting of, or consisting essentially of administering to the subject a therapeutically effective amount of a chemically defined reprogramming media such that the media contacts the fibroblast for a sufficient amount of time that the cardiofibroblast is reprogrammed into a cardiomyocyte. An exemplary media comprising advanced-DMEM/F12 media, 0.2% bovine serum albumin, 1× insulin-transferrin-selenium, 1× L-glutamine, and 250 μM ascorbic acid. In some embodiments, the methods further comprise transfecting the cardiofibroblast with at least one miRNA capable of facilitating the reprogramming a cardiofibroblast into a cardiomyocyte prior to contacting the cardiofibroblast with the media. In certain embodiments, the miRNA is selected from the group consisting of miR-1, miR-133, miR-208, miR-499 and combinations thereof.

In yet another aspect, provided herein are methods for reprogramming a cell into a cardiomyocyte in a subject in need thereof comprising: (a) removing at least one cell from the subject; (b) contacting the at least one cell with a chemically defined reprogramming media in an amount and time sufficient to reprogram the cell into a cardiomyocyte, the media comprising advanced-DMEM/F12 media, bovine serum albumin, insulin-transferrin-selenium, L-glutamine, and ascorbic acid; and (c) administering the newly reprogrammed cardiomyocyte to the subject. In some embodiments, the cell is selected from the group consisting of fibroblasts, adipocytes, or CD34+ umbilical cord blood cells. In some embodiments, the fibroblast is a cardiofibroblast. In some embodiments, the cell comprises cardiac fibrotic tissue. In some embodiments of any of the embodiments disclosed herein, the method further comprises transfecting the cell with at least one miRNA capable of facilitating the reprogramming the cell into a cardiomyocyte prior to contacting the cell with the media. In some embodiments, the miRNA is selected from the group consisting of miR-1, miR-133, miR-208, miR-499 and combinations thereof. In some embodiments of any of the embodiments disclosed herein, the method further comprises contacting the cell with a reprogramming efficiency-enhancing molecule. In some embodiments, the molecule is one or more molecules selected from the group consisting of valproic acid, bone, morphogenetic protein 4 (BMP4), JAK inhibitor 1, RG108, R(+)Bay K 8644, PS48, and A83-01. In some embodiments of any of the embodiments disclosed herein, the cardiomyocyte is characterized by an increased expression of a cardiomyocyte marker protein after the contacting step compared to the level of the marker protein before the contacting step. In some embodiments, the marker protein is selected from the group consisting of Nanog, Oct3, Sox2, Klf4, Hand2, Tbx5, Mesp1, Mef2c, Tnni3, Actn2, Nkx.2.5, aMHC, Cacnalc, Sen5a. In some embodiments of any of the embodiments disclosed herein, the newly reprogrammed cardiomyocyte is administered directly into the myocardium, e.g., in a region of damage or scarring. The cells are administered during surgery, e.g., open chest surgery, or by direct injection into the heart through the chest wall, or by catheter or stent. For example, the device is coated with ITS and/or ascorbic acid or a solution containing one or both compositions. In some embodiments, said fibrotic tissue is present in a heart diagnosed as comprising myocardial infarction, ischemic heart disease, hypertrophic cardiomyopathy, valvular heart disease, or congenital cardiomyopathy.

Yet another embodiment of the present disclosure provides a kit for the reprogramming of cardiac fibroblasts into cardiomyocytes in a subject, the kit comprising, consisting of, or consisting essentially of an amount of chemically defined reprogrammed media according to the present disclosure, a means of administering the media to a subject, and instructions for using the kit components. In some embodiments, the kit further comprises cell culture equipment, a means of removing cardiac fibroblasts from a subject, and a means for readministering the reprogrammed cells to the subject. In some embodiments of any of the embodiments disclosed herein, the kit further comprises a reprogramming efficiency-enhancing molecule. In some embodiments, the molecule is a direct regrogramming microRNA or is one or more molecules selected from the group consisting of valproic acid, bone morphogenetic protein 4 (BMP4), JAK inhibitor 1, RG108, R(+)Bay K 8644, PS48, and A83-01.

Another aspect of the present disclosure provides all that is described and illustrated herein.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts a diagram demonstrating the method of assessing direct reprogramming of fibroblasts to cardiomyocytes in accordance with one embodiment of the present disclosure.

FIGS. 2A-B are graphs showing the results of flow cytometric and qPCR analysis demonstrating that tail-tip fibroblasts express cardiac markers when cultured in chemically defined reprogramming media. (FIG. 2A) Tail-tip fibroblasts were cultured in either growth media (GM) or chemically defined reprogramming media (RM) for 14 days. Protein expression of the cardiac specific markers α-sarcomeric actinin, α-myosin heavy chain and cardiac troponin-T was determined by flow cytometry. P-values indicated. (FIG. 2B) Tail-tip fibroblasts were cultured in either growth media (GM) or chemically defined reprogramming media (RM) for the indicated times. Gene expression of the cardiac specific markers was determined by qPCR.

FIGS. 3A-B are graphs showing that reprogramming media augments the effect of miR combo in neonatal cardiac fibroblasts. (FIG. 3A) Neonatal cardiac fibroblasts were transfected with vehicle, negative control miR (Neg-miR) or miR combo. The day after transfection the cells were cultured in either growth media (GM) or reprogramming media (RM) for the indicated times. Expression of cardiac markers was determined by qPCR. N=3-7. *Comparisons made between miR combo and negative control miR***P<0.001, **P<0.01, *P<0.05. †Comparisons made between reprogramming media and growth media for each group †††P<0.001, ††P<0.01, †P<0.05. (FIG. 3B) Neonatal cardiac fibroblasts were cultured in either growth media (GM) or reprogramming media (RM) for 14 days. Protein expression of the cardiac specific markers α-myosin heavy chain (aMHC) and cardiac troponin-T (cTn-T) was determined by flow cytometry. N≥6. ***P<0.001, *P<0.05.

FIGS. 4A-D are graphs showing that chemically defined reprogramming media augments miR-combo reprogramming (FIG. 4A) Neonatal cardiac fibroblasts were cultured in either growth media [GM] or chemically defined reprogramming media [RM] for 7 and 14 days. Expression of the indicated cardiac specific genes was determined by qPCR. Expression in growth media was taken to be 1. (FIG. 4B) Neonatal cardiac fibroblasts were cultured in either growth media [GM] or chemically defined reprogramming media [RM] for 14 days. Expression of a-myosin heavy chain and cardiac troponin-T protein was determined by flow cytometry. (FIGS. 4C & D) Neonatal cardiac fibroblasts were cultured in either growth media [GM] or chemically defined reprogramming media [RM] for 7 (FIG. 4C) and 14 (FIG. 4D) days. Expression of the indicated genes was determined by qPCR, expression in cells cultured in GM and transfected with neg-miR was taken to be 1. * significant difference between miR-combo and neg-miR, † significant difference in the ratio miR-combo/neg-miR between GM and RM.

FIGS. 5A-B are graphs showing that reprogramming media induces cardiac gene expression in neonatal tail-tip fibroblasts. (FIG. 5A) Neonatal tail-tip fibroblasts were cultured in either growth media (GM) or reprogramming media (RM) for 14 (Cardiac Troponin-I) or 21 days (Scn5a, Cacnalc). Expression of the cardiac specific markers was determined by qPCR. N=3-11. ***P<0.001, **P<0.01, *P<0.05. (FIG. 5B) Neonatal tail-tip fibroblasts were cultured in either growth media (GM) or reprogramming media (RM) for 14 days. Protein expression of the cardiac specific markers α-sarcomeric actinin, α-myosin heavy chain and cardiac troponin-T was determined by flow cytometry. N=3.

FIGS. 6A-C are graphs showing that reprogramming media induces pluripotency gene expression. Neonatal tail-tip fibroblasts were cultured in growth media (GM) or reprogramming media (RM) for the indicated times. qPCR was used to determine the expression of (FIG. 6A) cardiac progenitor, (FIG. 6B) cardiac commitment, and (FIG. 6C) pluripotency markers. N=3-12. ***P<0.001, **P<0.01, *P<0.05.

FIGS. 7A-B are graphs showing that chemically defined reprogramming media promotes the expression of pluripotency markers. (FIG. 7A) Tail-tip fibroblasts were cultured in either growth media [GM] or chemically defined reprogramming media [RM] for the indicated times. Expression of the pluripotency markers Nanog, Oct4 and Rex-1 was determined by qPCR. Expression of cells cultured in GM for 1 day was taken to be 1. (FIG. 7B) Tail-tip fibroblasts were cultured in either growth media [GM] or chemically defined reprogramming media [RM] for indicated times. Expression of SSEA-1 and Oct4 protein was determined by flow cytometry.

FIGS. 8A-E are graphs showing that Nanog knockdown inhibits the effect of reprogramming media. (FIG. 8A) Neonatal cardiac fibroblasts were transfected with vehicle, negative control miR (Neg-miR) or miR combo. The day after transfection the cells were cultured in either growth media (GM) or reprogramming media (RM) for the indicated times. Nanog expression was determined by qPCR. N=3. *Comparisons made between miR combo and negative control miR **P<0.01, *P<0.05. †Comparisons made between reprogramming media and growth media for each group †††P<0.001, ††P<0.01, †P<0.05. (FIG. 8B) Neonatal tail-tip fibroblasts were transfected with a negative control or Nanog siRNA. Nanog expression was determined by qPCR three days after transfection. (FIGS. 8C-E) The effect of Nanog knockdown upon pluripotency, cardiac commitment and cardiac progenitor marker expression was determined by qPCR. N=3. ***P<0.001, **P<0.01, *P<0.05.

FIGS. 9A-B are graphs showing that insulin-transferrin-selenium is the active component of reprogramming media. (FIG. 9A) Neonatal tail-tip fibroblasts were cultured in the indicated medias for 14 days. Cardiac gene expression was determined by qPCR. N=3. ***P<0.001. (FIG. 9B) Neonatal tail-tip fibroblasts were cultured in reprogramming media lacking insulin-transferrin selenium (ITS) or reprogramming media lacking ITS with ITS added exogenously for 14 days. N=3. ***P<0.001.

### DETAILED DESCRIPTION

Insulin-transferrin-selenium was found to reprogram fibroblast into heart muscle cells through what is called a pluripotent state. Thus a cell culture reprogramming media (RM) containing Insulin-transferrin-selenium was developed. The most distinguishing feature of this a media formulation is that that it induces change of one type of somatic cell into another type of somatic cell. Current technologies require manipulations with viruses or reagents that are very toxic. An important advantage of this media is that it avoids such toxic components and greatly simplifies the process of cell reprogramming. Changing somatic cells from one type into another is important clinically, e.g, to repair and regenerate injured, damaged, or scarred tissue.

Following a heart attack, muscle cells (one type of somatic cell) die in large numbers and they are not replaced. Instead fibroblasts (another type of somatic cell) invade the injury zone and through a number of processes impair cardiac function. If these fibroblasts could be converted into heart muscle this would improve heart function. The media and methods of the invention are useful to reprogram fibroblasts into cardiomyocytes which function and behave accordingly, e.g., beating heart cells.

There are two approaches to generate cardiomyocytes from cells such as fibroblasts. Indirect reprogramming involves an intermediary step, fibroblasts are first converted to a pluripotent state, e.g., induced pluripotent stem (iPS) cells and these iPS cells are then differentiated into cardiomyocytes. Direct reprogramming does not require an intermediary step and the fibroblasts are directly converted into cardiomyocytes. Distinct combinations of transcription factors or microRNAs have been identified that directly reprogram fibroblasts. For example, a combination of microRNAs, (“miR” such as, but not limited to, miR-1, miR-133, miR-208, miR-499), have been shown to directly reprogram cardiac fibroblasts into cardiomyocytes both in vitro and in vivo (see U.S. Patent Application Publication No. 20140011281, the disclosure of which is incorporated by reference herein). Protocols that promote the in vitro differentiation of iPS cells into neonatal-like cardiomocytes are well established and relatively efficient. In comparison, direct reprogramming in vitro, whether by microRNAs or transcription factors, can be inefficient and induces a relatively low level of cardiac gene expression in cells such as fibroblasts. The media and methods described herein include those that contain ITS and induce/promote fibroblast to cardiomyocyte reprogramming as well as media and methods that include both ITS and ascorbic acid, thereby promoting both indirect and direct reprogramming of the cells without the use of viruses or toxic reagents.

Any cell culture media is augments with ITS and/or ascorbic acid to promote somatic cell reprogramming Cell culture media formulations are well known in the art and any one of the standard media preparations below are modified according to the invention to reprogram cells to a cardiomyocyte phenotype. Such media formulations are commercially available from vendors such as Sigma, ATCC, and Life Technologies.

Eagle's Minimum Essential Medium (EMEM) was among the first widely used media and was formulated by Harry Eagle from a simpler basal medium (BME). EMEM contains balanced salt solution, nonessential amino acids, and sodium pyruvate. It is formulated with a reduced sodium bicarbonate concentration (1500 ml/l) for use with 5% CO₂. Since EMEM is a non-complex medium, it is generally fortified with additional supplements or higher levels of serum making it suitable for a wide range of mammalian cells.

Dulbecco's Modified Eagle's Medium (DMEM) has almost twice the concentration of amino acids and four times the amount of vitamins as EMEM, as well as ferric nitrate, sodium pyruvate, and some supplementary amino acids. The original formulation contained 1,000 mg/L of glucose and was first reported for culturing embryonic mouse cells. A further variation with 4500 mg/L of glucose has been proved to be optimal for culture of various types of cells. DMEM is a basal medium and contains no proteins or growth promoting agents. Therefore, it requires supplementation to be a “complete” medium. It is most commonly supplemented with 5-10% Fetal Bovine Serum (FBS). DMEM utilizes a sodium bicarbonate buffer system (3.7 g/L) and therefore requires artificial levels of CO2 to maintain the required pH. Powdered media is formulated without sodium bicarbonate because it tends to gas off in the powdered state. Powdered media requires the addition of 3.7 g/L of sodium bicarbonate upon dissolving it in water. DMEM was used initially for the culture of mouse embryonic stem cells. It has been found to be widely applicable in primary mouse and chicken cells, viral plaque formation and contact inhibition studies.

RPMI-1640 is a general purpose media with a broad range of applications for mammalian cells, especially hematopoietic cells. RPMI-1640 was developed at Roswell Park Memorial Institute (RPMI) in Buffalo, N.Y. RPMI-1640 is a modification of McCoy's 5A and was developed for the long-term culture of peripheral blood lymphocytes. RPMI-1640 uses a bicarbonate buffering system and differs from the most mammalian cell culture media in its typical pH 8 formulation. RPMI-1640 supports the growth of a wide variety of cells in suspension and grown as monolayers. If properly supplemented with serum or an adequate serum replacement, RPMI-1640 has a wide range of applications for mammalian cells, including the culture of fresh human lymphocytes, fusion protocols, and growth of hybrid cells.

Ham's Nutrient Mixtures were originally developed to support the clonal outgrowth of Chinese hamster ovary (CHO) cells. There has been numerous modifications to the original formulation including Hams's F-12 medium, a more complex formulation than the original F-10 suitable for serum-free propagation. Mixtures were formulated for use with or without serum supplementation, depending on the type of cells being cultured. Ham's F-10 has been shown to support the growth of human diploid cells and white blood cells for chromosomal analysis. Ham's F-12 has been shown to support the growth of primary rat hepatocytes and rat prostate epithelial cells. Ham's F-12 supplemented with 25 mM HEPES provides more optimum buffering. Coon's modification of Ham's F-12 contains of almost two times the amount of amino acids and pyruvate as compared to F-12 and also includes ascorbic acid. It was developed for culturing hybrid cells produced by viral fusion.

DMEM/F12 is a mixture of DMEM and Ham's F-12 and is an extremely rich and complex medium. It supports the growth of a broad range of cell types in both serum and serum-free formulations. HEPES buffer is included in the formulation at a final concentration of 15 mM to compensate for the loss of buffering capacity incurred by eliminating serum.

Iscove's Modified Dulbecco's Medium (IMDM) is a highly enriched synthetic media well suited for rapidly proliferating, high-density cell cultures. IMDM is a modification of DMEM containing selenium, and has additional amino acids, vitamins and inorganic salts as compared to DMEM. It has potassium nitrate instead of ferric nitrate and also contains HEPES and sodium pyruvate. It was formulated for the growth of lymphocytes and hybridomas. Studies have demonstrated that IMDM can support murine B lymphocytes, hemopoietic tissue from bone marrow, B cells stimulated with lipopolysaccharide, T lymphocytes, and a variety of hybrid cells.

The present invention is based, in part, on the observation that a media composed of a base cell culture media (such as, Advanced DMEM/F12), ascorbic acid, purified BSA, glutamine, and insulin-transferrin-selenium, henceforth referred to as “reprogramming media (RM),” directly reprograms cells (such as fibroblasts) into cardiomyocytes as well as augments miR-directed reprogramming of fibroblasts into cardiomyocytes. Using the instantly described compositions and methods, the efficiency of cell to cardiomyocyte reprograming was increased by a surprising 5 to 15-fold depending upon the cardiac marker tested. RM induced the expression of cardiac genes in neonatal tail-tip and cardiac fibroblasts. Moreover, RM strongly induced the expression of the pluripotency markers Nanog, Oct4, Sox2, and Klf4, with miR combo augmenting the effect. Knockdown of Nanog by siRNA inhibited the effect of RM on cardiac gene expression. Removal of insulin-transferrin-selenium completely inhibited the effect of reprogramming media upon cardiac gene expression. Accordingly, the compositions and methods described herein have the potential to significantly improve the efficiency of direct reprogramming of cells (such as fibroblasts, for example cardiac fibroblasts) to cardiomyocytes following injury to heart tissue brought on by conditions such as, but not limited to, myocardial infarction, ischemic heart disease, hypertrophic cardiomyopathy, valvular heart disease, or congenital cardiomyopathy.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### I. Definitions

As used herein, the term “GM” or “fibroblast growth media” refers to the media used to culture isolated fibroblasts. An exemplary GM comprises the following: DMEM (ATCC#30-2002), 15% v/v embryonic stem cell qualified fetal bovine serum, and 1× penicillin/streptomycin. As used herein, the terms “chemically defined reprogramming media”, “chemically defined media” and “RM” are used interchangeably herein and refer to that media used when reprogramming the cardiofibroblasts. In one embodiment, the chemically defined reprogramming media comprises the following: Advanced-DMEM/F12 media, 0.2% bovine serum albumin, 1× insulin-transferrin-selenium, 1× L-glutamine, 250 μM ascorbic acid.

As used herein, a “purified” protein refers to a protein (such as bovine serum albumin) that has been separated from all other cellular and serum components, such as, but not limited to, other proteins, carbohydrates, lipids, cholesterol, etc. “Purified” proteins have been purified to a level of purity not found in nature and are suitable for clinical use and in the context of ex vivo cell therapy. Purity is measured by any appropriate standard method, for example, by column chromatography, thin layer chromatography, or high-performance liquid chromatography (HPLC) analysis.

All polynucleotides (e.g., microRNAs), polypeptides, amino acids, or other compositons or compounds used in the cell culture medium of the invention are purified and/or isolated. Specifically, as used herein, an “isolated” or “purified” nucleic acid molecule is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents.

As used herein, the term “at least one miRNA” or “miR-combo” refers to any mixture (e.g., one or more than one) of miRNA molecules capable of facilitating the reprogramming of cells (such as, but not limited fibroblasts, for example cardiofibroblasts) into cardiomyocytes. Suitable miRs include, but are not limited to, miR-1, miR-133, miR-208 and miR-499

The terms “treating,” “treat,” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

By the terms “effective amount” and “therapeutically effective amount” of a formulation or formulation component is meant a sufficient amount of the formulation or component to provide the desired effect. For example, by “an effective amount” is meant an amount of a microRNA and/or reprogramming media to directly reprogram cells (such as fibroblasts, e.g., cardiac fibroblasts) to cardiomyocytes in a subject. Ultimately, the attending physician or veterinarian decides the appropriate amount and dosage regimen.

As described herein, small molecules include, but are not limited to, peptides, peptidomimetics (e.g., peptoids), amino acids, amino acid analogs, polynucleotides, polynucleotide analogs, nucleotides, nucleotide analogs, organic and inorganic compounds (including heterorganic and organomettallic compounds) having a molecular weight less than about 5,000 grams per mole, organic or inorganic compounds having a molecular weight less than about 2,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. A small molecule inhibitor is a compound that is less than 2000 daltons in mass. The molecular mass of the inhibitory compounds is preferably less than 1000 daltons, more preferably less than 600 daltons, e.g., the compound is less than 500 daltons, 400 daltons, 300 daltons, 200 daltons, or 100 daltons.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, mice, chickens, amphibians, reptiles, and the like. Preferably, the subject is a human patient. More preferably, the subject is a human patient who has suffered damage to the cardiac tissue, such as that resulting from a myocardial infarction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

### II. Compositions of the Invention

A. Reprogramming Media

Provided herein is a chemically defined reprogramming media (RM) that reprograms cells (such as, fibroblasts) into cardiomyocytes and/or which enhances the efficiency of miR-mediated direct reprogramming of cells into cardiomyocytes.

The RM includes a base tissue culture media such as those described above. Tissue culture is generally understood as the growth of eukaryotic cells in vitro. Tissue culture media and processes are well known (see e.g., Helgason and Miller 2004 Basic Cell Culture Protocols, 3d Ed., Humana Press, ISBN-10 1588292843; Vunjak-Nokakovic and Freshney, ed. 2006 Culture of Cells for Tissue Engineering, Wiley-Liss, ISBN-10 0471629359; Freshney 2005 Culture of Animal Cells, 5th ed., Wiley-Liss, ISBN-10 0471453293.) Therefore, compositions and methods of the present invention can utilize any base tissue culture media capable of maintaining eukaryotic cells in culture for extended periods of time. In one embodiment, the base tissue culture media comprises Dulbecco's Modified Eagle Medium: Nutrient Mixture F-12 media (advanced-DMEM/F12), which is commercially available from Life Technologies. In another embodiment, the base tissue culture media is free of serum.

The RM may also contain purified bovine serum albumin (also known as BSA or “Fraction V”) which is a serum albumin protein derived from cows commonly used in cell culture media. The RM can contain from about 0.05% to 0.4% w/v purified BSA, such as any of about 0.075%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, or more, w/v purified BSA inclusive of all values falling within these percentages.

The RM further contains insulin-transferrin-selenium (ITS) as the active composition that mediates reprogramming. This reagent is commercially available through companies such as Gibco®. Prior to the invention, ITS was used as a basal medium supplement in order to reduce the amount of fetal bovine serum (FBS) needed to culture cells. Insulin promotes glucose and amino acid uptake, lipogenesis, intracellular transport, and the synthesis of proteins and nucleic acids. Transferrin is an iron carrier and it may also help to reduce toxic levels of oxygen radicals and peroxide. Selenium, (for example, in the form of sodium selenite), is a co-factor for glutathione peroxidase and other proteins, and is used as an antioxidant in media. The data described herein demonstrates that ITS alone in a culture media induces efficient reprogramming of fibroblasts to cardiomyocytes after only days or a week of culture. In some embodiments, the RM contains from about 0.1× to 3× ITS, such as any of about 0.1×, 0.2×, 0.3×, 0.4×, 0.5×, 0.6×, 0.7×, 0.8×, 0.9×, 1×, 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, or more ITS, inclusive of values falling in between these numbers. ITS is frequently sold as a 100× stock solution (see table below). Each 10 mL vial of Insulin-Transferrin-Selenium 100× Supplement is sufficient for up to one liter of medium. In general, it is necessary to add 2-4% PBS to achieve optimal growth, although some adherent cultures may require less serum supplementation following initial adaptation. Store Insulin-Transferrin-Selenium supplemented medium in the dark at 2° C. to 8° C.

Formulation

| | Concentration (g/L) | | |
|---|---|---|---|
| Component | ITS | ITS-A | ITS-X |
| Insulin | 1.00 | 1.00 | 1.00 |
| Transferrin | 0.55 | 0.55 | 0.55 |
| Sodium Selenite | 0.00067 | 0.00067 | 0.00067 |
| Sodium Pyruvate | — | 11.00 | — |
| Ethanolamine | — | — | 0.20 |

In some embodiments, the ratio of insulin to selenium to transferrin is 1493:1:821, respectively.

Regarding the selenium component of ITS, it can be present at a concentration of about 1 μg/L to about 100 μg/L, such as any of about 1 μg/L, 2 μg/L, 3 μg/L, 4 μg/L, 5 μg/L, 6 μg/L, 7 μg/L, 8 μg/L, 9 μg/L, 10 μg/L, 11 μg/L, 12 μg/L, 13 μg/L, 14 μg/L, 15 μg/L, 16 μg/L, 17 μg/L, 18 μg/L, 19 μg/L, 20 μg/L, 21 μg/L, 22 μg/L, 23 μg/L, 24 μg/L, 25 μg/L, 26 μg/L, 27 μg/L, 28 μg/L, 29 μg/L, 30 μg/L, 31 μg/L, 32 μg/L, 33 μg/L, 34 μg/L, 35 μg/L, 36 μg/L, 37 μg/L, 38 μg/L, 39 μg/L, 40 μg/L, 41 μg/L, 42 μg/L, 43 μg/L, 44 μg/L, 45 μg/L, 46 μg/L, 47 μg/L, 48 μg/L, 49 μg/L, 50 μg/L, 55 μg/L, 60 μg/L, 65 μg/L, 70 μg/L, 75 μg/L, 80 μg/L, 85 μg/L, 90 μg/L, 95 μg/L, or 100 μg/L, or more selenium, inclusive of all values falling in between these concentrations. In some embodiments, selenium is present in ITS at a concentration of about 6.7 μg/L.

Regarding the insulin component of ITS, it can be present at a concentration of about 10 nM to about 10 mM, such as any of about 10 nM, 25 nM, 50 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM, 105 nM, 106 nM, 107 nM, 108 nM, 109 nM, 110 nM, 111 nM, 112 nM, 113 nM, 114 nM, 115 nM, 116 nM, 117 nM, 118 nM, 119 nM, 120 nM, 121 nM, 122 nM, 123 nM, 124 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 600 nM, 700 nM, 800 nM, 900 nM, 1 mM, 2 mM, 3 mM, 4 mM, 5 mM, 6 mM, 7 mM, 8 mM, 9 mM, 10 mM, or more insulin, inclusive of all values falling in between these concentrations. In some embodiments, insulin is present in ITS at a concentration of about 100 nM.

Regarding the transferrin component of ITS, it can be present at a concentration of about 0.002 g/L to about 1 g/L, such as any of about 0.002 g/L g/L, 0.01 g/L, 0.015 g/L, 0.02 g/L, 0.03 g/L, 0.04 g/L, 0.045 g/L, 0.046 g/L, 0.047 g/L, 0.048 g/L, 0.049 g/L, 0.05 g/L, 0.051 g/L, 0.052 g/L, 0.053 g/L, 0.054 g/L, 0.055 g/L, 0.056 g/L, 0.057 g/L, 0.058 g/L, 0.059 g/L, 0.06 g/L, 0.061 g/L, 0.062 g/L, 0.063 g/L, 0.064 g/L, 0.065 g/L, 0.07 g/L, 0.08 g/L, 0.09 g/L, 0.1 g/L, 0.2 g/L, 0.3 g/L, 0.4 g/L, 0.5 g/L, 0.6 g/L, 0.7 g/L, 0.8 g/L, 0.9 g/L, or 1 g/L transferrin or more, inclusive of all values falling in between these concentrations. In some embodiments, transferrin is present in ITS at a concentration of about 0.055 g/L.

With respect to ITS, the ratio of insulin to selenium can be from about 200-10000:1, such as any of about 300:1, 400:1, 500:1, 600:1, 700:1, 800:1, 900:1, 1100:1, 1200:1, 1300:1, 1400:1, 1500:1, 1600:1, 1700:1, 1800:1, 1900:1, 2000:1, 2100:1, 2200:1, 2300:1, 2400:1, 2500:1, 2600:1, 2700:1, 2800:1, 2900:1, 3000:1, 3100:1, 3200:1, 3300:1, 3400:1, 3500:1, 3600:1, 3700:1, 3800:1, 3900:1, 4100:1, 4200:1, 4300:1, 4400:1, 4500:1, 4600:1, 4700:1, 4800:1, 4900:1, 5000:1, 5250:1, 5500:1, 5750:1, 6000:1, 6250:1, 6500:1, 6575:1, 7000:1, 7250:1, 7500:1, 7750:1, 8000:1, 8250:1, 8500:1, 8575:1, 9000:1, 9250:1, 9500:1, 9750:1, or 10,000:1, inclusive of all ratios falling within these values. In some embodiments, the ratio of insulin to selenium is 1493:1.

Also with respect to ITS, the ratio of transferrin to selenium can be from about 50-5000:1, such as any of about 100:1. 200:1, 300:1, 400:1, 500:1, 600:1, 700:1, 800:1, 900:1, 1100:1, 1200:1, 1300:1, 1400:1, 1500:1, 1600:1, 1700:1, 1800:1, 1900:1, 2000:1, 2100:1, 2200:1, 2300:1, 2400:1, 2500:1, 2600:1, 2700:1, 2800:1, 2900:1, 3000:1, 3100:1, 3200:1, 3300:1, 3400:1, 3500:1, 3600:1, 3700:1, 3800:1, 3900:1, 4100:1, 4200:1, 4300:1, 4400:1, 4500:1, 4600:1, 4700:1, 4800:1, 4900:1, or 5000:1, inclusive of all ratios falling within these values. In some embodiments, the ratio of transferrin to selenium is 821:1.

Further with respect to ITS, the ratio of insulin to transferrin can be from about 1.2-12:1, such as any of about 1.3:1, 1.4:1, 1.5:1, 1.6:1, 1.7:1, 1.8:1, 1.9:1, 2:1, 2.2:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1. 7:1, 7.1:1, 7.2:1, 7.3:1, 7.4:1, 7.5:1, 7.6:1, 7.7:1, 7.8:1, 7.9:1, 8:1, 8.2:1, 8.3:1, 8.4:1, 8.5:1, 8.6:1, 8.7:1, 8.8:1, 8.9:1, 9:1, 9.1:1. 9.2:1. 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.4:1, 11.4:1, 11.5:1, 11.6:1, 11.7:1, 11.8:1, 11.9:1, or 12:1, inclusive of all ratios falling within these values. In some embodiments, the ratio of insulin to transferrin is 1.8:1.

The RM can also include L-glutamine, which is commonly used in cell culture media. In some embodiments, the RM contains from about 0.1× to 3× L-glutamine, such as any of about 0.1×, 0.2×, 0.3×, 0.4×, 0.5×, 0.6×, 0.7×, 0.8×, 0.9×, 1×, 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, or more L-glutamine, inclusive of values falling in between these numbers. For example, L-glutamine is present at a concentration of 0.2 mM to 20 mM, 0.5 mM to 15 mM. For example, the RM comprises 2 mM L-glutamine.

The RM additionally contains ascorbic acid. Ascorbic acid is a naturally occurring organic compound with antioxidant properties. It dissolves well in water to give mildly acidic solutions and is one form (“vitamer”) of vitamin C In some embodiments, the RM contains from about 50 μM to 50 mM ascorbic acid, such as any of about 50 μM, 55 μM, 60 μM, 65 μM, 70 μM, 75 μM, 80 μM, 85 μM, 90 μM, 95 μM, 100 μM, 105 μM, 110 μM, 115 μM, 120 μM, 125 μM, 130 μM, 135 μM, 140 μM, 145 μM, 150 μM, 155 μM, 160 μM, 165 μM, 170 μM, 175 μM, 180 μM, 185 μM, 190 μM, 195 μM, 200 μM, 205 μM, 210 μM, 215 μM, 220 μM, 225 μM, 230 μM, 235 μM, 240 μM, 245 μM, 250 μM, 255 μM, 260 μM, 265 μM, 270 μM, 275 μM, 280 μM, 285 μM, 290 μM, 295 μM, 300 μM, 305 μM, 310 μM, 315 μM, 320 μM, 325 μM, 330 μM, 335 μM, 340 μM, 345 μM, 350 μM, 355 μM, 360 μM, 365 μM, 370 μM, 375 μM, 380 μM, 385 μM, 390 μM, 395 μM, 400 μM, 405 μM, 410 μM, 415 μM, 420 μM, 425 μM, 430 μM, 435 μM, 440 μM, 445 μM, 450 μM, 455 μM, 460 μM, 465 μM, 470 μM, 475 μM, 480 μM, 485 μM, 490 μM, 495 μM, 500 μM, 600 μM, 700 μM, 800 μM, 900 μM, 1 mM, 5 mM, 10 mM, 15 mM, 20 mM, 25 mM, 30 mM, 35 mM, 40 mM, 45 mM, 50 mM or more ascorbic acid, inclusive of all values falling in between these concentrations.

B. Cells Cultured in Reprogramming Media

Reprogramming is process by which cells change phenotype, state of differentiation, or function. This process is exploited as a tool for creating patient-specific pluripotent cells that are useful in cell replacement therapies. As discussed above, in “direct reprogramming,” the differentiated state of a specialized somatic cell is reversed to another type (e.g., endocrine cells to exocrine cells or fibroblasts to neurons or, as described herein, cardiomyocytes). Accordingly, provided herein are compositions comprising cells cultured in any of the RMs described herein. Suitable cells for reprogramming include adipocytes, CD34+ cord blood cells, and fibroblasts (such as cardiac or dermal fibroblasts).

Adipocytes are an exemplary population for reprogramming Adipocytes, also known as lipocytes and fat cells, are the cells that primarily compose adipose tissue, specialized in storing energy as fat. Although the lineage of adipocytes is still unclear, preadipocytes are undifferentiated fibroblasts that can be stimulated to form adipocytes. CD34+ cord blood cells are also an exemplary population for reprogramming CD34+ cells are hematopoietic stem cells present in umbilical cord blood.

Optionally, fibroblasts are the starting population for reprogramming Fibroblasts are traditionally defined as cells of mesenchymal origin that produce interstitial collagen (in contrast to myocytes that form collagen type IV as part of their basement membrane, fibroblasts also produce types I, III and VI). In general, fibroblasts lack a basement membrane and tend have multiple processes or sheet-like extensions. They contain an oval nucleus (with 1 or 2 nucleoli), extensive rough endoplasmic reticulum, a prominent Golgi apparatus, and abundant cytoplasmic granular material. Specific markers are scarce; however, DDR2 is useful as a marker. This marker is expressed in fibroblasts and other cells but not other cardiac cells. The mesenchymal cells that form the cardiac fibroblast population are believed to be derived from two principal sources: (1) the pro-epicardial organ, and (2) the epithelial-mesenchymal transformation during the formation of cardiac valves.

C. miRNAs

Also provided herein are compositions comprising cells cultured in any of the RMs described herein, wherein the cells have been transfected with one or more nucleic acids encoding a microRNA (miRNA). The microRNA oligonucleotides lead to transient overexpression of the desired microRNA in the target cell or tissue. Thus, the oligonucleotide increases the level of an endogenous microRNA sequence. Similarly, administration of microRNA delivery constructs such as lentiviruses lead to expression of microRNAs (stem loop sequence or mature sequence) in the cells. Preferred mir oligonucleotides (or corresponding miR-expressing delivery constructs) are selected from the group consisting of mir1, mir133 (or mir133a), mir138, mir206, mir208, mir499, and mir126 as well as the following combinations: mir1; mir1, mir133a, mir208; mir1, mir133a, mir206; mir1, mir133a, mir208, mir499-5p, mir1, mir133a, mir206, mir499-5p; mir1, mir133; mir1, mir138; mir1, mir206; mir1, mir208; mir133, mir138; mir133, mir206; mir133, mir208; mir138, mir206; mir138, mir208; mir206, mir208; mir1, mir138, mir208; mir1, mir206, mir208; mir138, mir206, mir208; mir1, mir133, mir206; mir1, mir133, mir208; mir1, mir138, mir206; mir133, mir138, mir208; and mir133, mir138, mir206. Preferred oligonucleotide compositions include the combination of 1, 133a, and 206; the combination of 1, 133a, and 208; the combination of 1, 206, and 208; the combination of 1, 133a, 208, and 499-5p; the combination of 1, 133a, 206, and 499-5p; 1; 206; as well as the combination of mir1, mir138, and mir208. The compositions are introduced into a cell by any method known to preserve the viability of the cell, e.g., transfection or transduction. Transfection is the process of introducing nucleic acids into cells by non-viral methods, and transduction is the process whereby foreign DNA is introduced into another cell via a viral vector.

Nucleotide sequences of these preferred oligonucleotide constructs or combinations of constructs (and their corresponding mature forms) are listed below. Exemplary oligomeric compounds (stem-loop precursors) range in size from 50-90 nucleotides in length (or any length within that range, with an average length of approximately 70 nucleotides), and exemplary mature oligonucleotide compounds are 17 to 25 subunits in length, e.g., oligomeric compounds are 17, 18, 19, 20, 21, 22, 23, 24 or 25 subunits in length. For example, a stem-loop precursor is approximately 70 nucleotides and the mature nucleotide product is approximately 22 nucleotides in length. The uncapitalized “mir-” refers to the pre-miRNA, while a capitalized “miR-” refers to the mature form. A pre-microRNA comprises a stem-loop secondary structure.

Mmu-miR-1
STEM-LOOP
(SEQ ID NO: 1)
GCUUGGGACACAUACUUCUUUAUAUGCCCAUAUGAACCUGCUAAGCUAUG
GAAUGUAAAGAAGUAUGUAUUUCAGGC
MATURE
(SEQ ID NO: 2)
UGGAAUGUAAAGAAGUAUGUAU
Mmu-miR-133a
STEM-LOOP
(SEQ ID NO: 3)
GCUAAAGCUGGUAAAAUGGAACCAAAUCGCCUCUUCAAUGGAUUUGGUCC
CCUUCAACCAGCUGUAGC
MATURE
(SEQ ID NO: 4)
UUUGGUCCCCUUCAACCAGCUG
Mmu-miR-206
STEM-LOOP
(SEQ ID NO: 5)
CCAGGCCACAUGCUUCUUUAUAUCCUCAUAGAUAUCUCAGCACUAUGGAA
UGUAAGGAAGUGUGUGGUUUUGG
MATURE
(SEQ ID NO: 6)
UGGAAUGUAAGGAAGUGUGUGG
Mmu-miR-208a
STEM-LOOP
(SEQ ID NO: 7)
UUCCUUUGACGGGUGAGCUUUUGGCCCGGGUUAUACCUGACACUCACGUA
UAAGACGAGCAAAAAGCUUGUUGGUCAGAGGAG
MATURE
(SEQ ID NO: 8)
AUAAGACGAGCAAAAAGCUUGU
Human miR-1-1
STEM-LOOP
(SEQ ID NO: 9)
UGGGAAACAUACUUCUUUAUAUGCCCAUAUGGACCUGCUAAGCUAUGGAA
UGUAAAGAAGUAUGUAUCUCA
Human miR-1-2
STEM-LOOP
(SEQ ID NO: 10)
ACCUACUCAGAGUACAUACUUCUUUAUGUACCCAUAUGAACAUACAAUGC
UAUGGAAUGUAAAGAAGUAUGUAUUUUUGGUAGGC
MATURE SEQUENCE FOR BOTH miR1 STEM-LOOPS:
(SEQ ID NO: 11)
UGGAAUGUAAAGAAGUAUGUAU
Human miR-133a
Human miR-133a-1
STEM-LOOP
(SEQ ID NO: 12)
ACAAUGCUUUGCUAGAGCUGGUAAAAUGGAACCAAAUCGCCUCUUCAAUG
GAUUUGGUCCCCUUCAACCAGCUGUAGCUAUGCAUUGA
Human miR-133a-2
STEM-LOOP
(SEQ ID NO: 13)
GGGAGCCAAAUGCUUUGCUAGAGCUGGUAAAAUGGAACCAAAUCGACUGU
CCAAUGGAUUUGGUCCCCUUCAACCAGCUGUAGCUGUGCAUUGAUGGCGC
CG
MATURE SEQUENCE FOR BOTH miR133a STEM LOOPS
(SEQ ID NO: 14)
UUUGGUCCCCUUCAACCAGCUG
Human miR-206
STEM-LOOP
(SEQ ID NO: 15)
UGCUUCCCGAGGCCACAUGCUUCUUUAUAUCCCCAUAUGGAUUACUUUGC
UAUGGAAUGUAAGGAAGUGUGUGGUUUCGGCAAGUG
MATURE SEQUENCE FOR miR-206
(SEQ ID NO: 16)
UGGAAUGUAAGGAAGUGUGUGG
Human miR-208a
STEM-LOOP
(SEQ ID NO: 17)
UGACGGGCGAGCUUUUGGCCCGGGUUAUACCUGAUGCUCACGUAUAAGAC
GAGCAAAAAGCUUGUUGGUCA
MATURE SEQUENCE FOR miR-208
(SEQ ID NO: 18)
AUAAGACGAGCAAAAAGCUUGU
Human miR-138-1
STEM-LOOP
(SEQ ID NO: 19)
CCCUGGCAUGGUGUGGUGGGGCAGCUGGUGUUGUGAAUCAGGCCGUUGCC
AAUCAGAGAACGGCUACUUCACAACACCAGGGCCACACCACACUACAGG
Human miR-138-2
STEM-LOOP
(SEQ ID NO: 20)
CGUUGCUGCAGCUGGUGUUGUGAAUCAGGCCGACGAGCAGCGCAUCCUCU
UACCCGGCUAUUUCACGACACCAGGGUUGCAUCA
MATURE SEQUENCE FOR BOTH miR-138-1 and miR-138-2
(SEQ ID NO: 21)
AGCUGGUGUUGUGAAUCAGGCCG
Human miR-499-5p
STEM-LOOP (MMu-miR-499)
(SEQ ID NO: 24)
GGGUGGGCAGCUGUUAAGACUUGCAGUGAUGUUUAGCUCCUCUGCAUGUG
AACAUCACAGCAAGUCUGUGCUGCUGCCU
MATURE (Mmu-miR-499/Hsa-miR-499-5p; sequence is
conserved)
(SEQ ID NO: 25)
UUAAGACUUGCAGUGAUGUUU

### D. Reprogramming Efficiency-Enhancing Molecules

Optionally, cells cultured in any of the RM compositions described herein can be transfected with one or more of any of the microRNAs described herein and further cultured with a small molecule or other agent (e.g., a recombinant protein) to increase reprogramming efficiencies. Small molecules suitable for increasing the efficiency of conversion to cardiac myocytes include valproic acid, bone morphogenetic protein 4 (BMP4), Janus protein tyrosine kinase (JAK) inhibitor 1, RG108, R(+)Bay K 8644, PS48, and A83-01. These agents are delivered (e.g., infused or injected) to the subject before, after, or together with miR oligonucleotides or microRNA-expressing viral constructs. In the case of ex vivo reprogramming, the agents are added to the cell culture media.

Valproic acid (VPA; 2-propylpentanoic acid; C8H16O2) is a chemical compound that has found clinical use as an anticonvulsant and mood-stabilizing drug, primarily in the treatment of epilepsy, bipolar disorder, and major depression and which can be added to any of the RM compositions described herein to increase reprogramming efficiency of target cells. Valproic acid also blocks the voltage-gated sodium channels and T-type calcium channels. These mechanisms make valproic acid a broad spectrum anticonvulsant drug. Serum or plasma valproic acid concentrations are generally in a range of 20-100 mg/L during controlled therapy. Valproic acid is available from Stemgent, and used at a final concentration of about 0.01 mM to about 10 mM, e.g., about 0.1 mM to about 5 mM or about 1 mM to about 3 mM. Preferably, valproic acid is used at a final concentration of about 2 mM. Valproic acid is administered in about one dose to about 5 doses, e.g., about 1 dose, about 2 doses, about 3 doses, about 4 doses, or about 5 doses. Preferably, valproic acid is administered in 2 doses. Valproic acid is administered about 1 hour to about 96 hours prior to miR transfection and about 1 hour to about 96 hours after miR transfection, e.g., about 12 hours to about 72 hours or about 24 hours to about 60 hours prior to and after miR transfection. Preferably, valproic acid is administered in two doses: one dose at 48 hours prior to miR transfection and one dose at 48 hours post-transfection.

Bone morphogenetic proteins (BMPs) are a group of growth factors also known as cytokines and as metabologens. Originally discovered by their ability to induce the formation of bone and cartilage, BMPs are now considered to constitute a group of pivotal morphogenetic signals, orchestrating tissue architecture throughout the body. Signal transduction through BMPRs results in mobilization of members of the SMAD family of proteins. The signaling pathways involving BMPs, BMPRs and Smads are important in the development of the heart, central nervous system, and cartilage, as well as post-natal bone development. BMP4 plays an important role in the onset of endochondral bone formation in humans. It is involved in muscle development, bone mineralization, and uteric bud development. BMP4 is also of crucial importance for cardiac development and differentiation. BMP-4 is available from Stemgent, and used at a final concentration of about 0.1 ng/mL to about 100 ng/mL, e.g., about 1 ng/mL to about 50 ng/mL or about 10 ng/mL to about 30 ng/mL. Preferably, BMP-4 is used at a final concentration of about 20 ng/mL. BMP-4 is administered every day beginning about 1 day to about 14 days before or after transfection of miRs, e.g., BMP-4 is administered about 2 days to about 13 days or about 5 days to about 10 days before or after transfection of miRs. Preferably, BMP-4 is administered 7 days post-transfection of miRs. Subsequently, BMP-4 is administered once/day for about 1 day, about 2 days, about 3 days, about 4 days, about 5 days, about 6 days, about 7 days, about 8 days, about 9 days, or about 10 days. Preferably, BMP-4 is administered every day for cells in culture.

JAK Inhibitor

1(2-(1,1-Dimethylethyl)-9-fluoro-3,6-dihydro-7H-benz[h]-imidaz [4,5-f]isoquinolin-7-one, Pyridone 6, P6, DBI (420099 JAK Inhibitor I); C18H16FN3O) is a potent, reversible, cell-permeable, and ATP-competitive inhibitor of Janus protein tyrosine kinases (JAKs). This molecule displays potent inhibitory activity against JAK1 (IC50=15 nM for murine JAK1), JAK2 (IC50=1 nM), JAK3 (Ki=5 nM), and Tyk2 (IC50=1 nM), and also inhibits other kinases at much higher concentrations. JAK inhibitor 1 also inhibits IL-2- and IL-4-dependent proliferation of CTLL cells and blocks the phosphorylation of STATS. This molecule also induces the growth inhibition of multiple myeloma cells expressing activated JAKs and STATS. The JAK inhibitor 1 is available from EMD Biosciences, and used at a final concentration of about 0.001 μM to about 10 μM, e.g., about 0.01 μM to about 5 μM or about 0.1 μM to about 1 μM. Preferably, the JAK inhibitor 1 is used at a final concentration of about 0.5 μM. The JAK inhibitor 1 is administered about 1 hour to about 96 hours before or after transfection of miRs, e.g., the JAK inhibitor 1 is administered once/day beginning about 12 hours to about 72 hours or about 24 hours to about 60 hours before or after transfection of miRs. Preferably, the JAK inhibitor 1 is administered 48 hours post-transfection of miRs. The JAK Inhibitor 1 is administered once/day for about 1 day, about 2 days, about 3 days, about 4 days, about 5 days, about 6 days, about 7 days, about 8 days, about 9 days, or about 10 days. Preferably, the JAK inhibitor 1 is administered every day for 5 days.

RG108

(2-(1,3-Dioxo-1,3-dihydro-2H-isoindol-2-yl)-3-(1H-indol-3-yl)propionic acid, N-Phthalyl-L-tryptophan; C19H14N2O4) is a potent and specific DNA methyltransferase (DNMT) inhibitor. It causes demethylation and reactivation of tumor suppressor genes and can be used to enhance reprogramming RG108 has been found to inhibit human tumor cell line proliferation and increases doubling time in culture. This molecule is soluble to 100 mM in DMSO and to 100 mM in ethanol. RG108 is available from Stemgent, and used at a final concentration of about 0.001 μM to about 10 μM, e.g., about 0.001 μM to about 5 μM or about 0.01 μM to about 0.1 μM. Preferably, RG108 is used at a final concentration of about 0.04 μM. RG108 is administered in about one dose to about 5 doses, e.g., about 1 dose, about 2 doses, about 3 doses, about 4 doses, or about 5 doses. Preferably, RG108 is administered in 2 doses. RG108 is administered about 1 hour to about 96 hours prior to miR transfection and about 1 hour to about 96 hours after miR transfection, e.g., about 12 hours to about 72 hours or about 24 hours to about 60 hours prior to and after miR transfection. Preferably, RG108 is administered in two doses: one dose at 48 hours prior to miR transfection and one dose at 48 hours post-transfection.

R(+)Bay K 8644 (R-(+)-1,4-Dihydro-2,6-dimethyl-5-nitro-4-[2-(trifluoromethyl)phenyl]-3-pyridinecarboxylic acid methyl ester; C16HisF3N2O4) is a L-type Ca²⁺-channel blocker with negative inotropic and vasodilatatory effects in vivo. This enantiomer has opposite effects to the racemate (±)-Bay K 8644 and (S)-(−)-enantiomer. In combination with BIX-01294, this molecule helps generate induced pluripotent stem cells (iPSCs) from mouse embryonic fibroblasts (MEFs). This molecule is soluble to 100 mM in ethanol and to 100 mM in DMSO. R(+)Bay K 8644 is available from Stemgent, and used at a final concentration of about 0.01 μM to about 10 μM, e.g., about 0.1 μM to about 5 μM or about 1 μM to about 3 Preferably, R(+)Bay K 8644 is used at a final concentration of about 2 R(+)Bay K 8644 is administered in about one dose to about 5 doses, e.g., about 1 dose, about 2 doses, about 3 doses, about 4 doses, or about 5 doses. Preferably, R(+)Bay K 8644 is administered in 2 doses. R(+)Bay K 8644 is administered about 1 hour to about 96 hours prior to miR transfection and about 1 hour to about 96 hours after miR transfection, e.g., about 12 hours to about 72 hours or about 24 hours to about 60 hours prior to and after miR transfection. Preferably, R(+)Bay K 8644 is administered in two doses: one dose at 48 hours prior to miR transfection and one dose at 48 hours post-transfection.

PS48 (5-(4-Chloro-phenyl)-3-phenyl-pent-2-enoic acid; C17H15C102) is a PDK1 (phosphoinositide-dependent protein kinase 1) activator which binds to the HM/PIF binding pocket rather than the ATP-binding site. PS48 is one of only a few truly allosteric compounds targeting a regulatory binding site on a protein kinase catalytic domain that is not adjacent to or overlapping with the ATP-binding site. This molecule is soluble in DMSO. PS48 is available from Stemgent, and used at a final concentration of about 0.01 μM to about 10 μM, e.g., about 0.1 μM to about 8 μM or about 4 μM to about 6 Preferably, PS48 is used at a final concentration of about 5 PS48 is administered in about one dose to about 5 doses, e.g., about 1 dose, about 2 doses, about 3 doses, about 4 doses, or about 5 doses. Preferably, PS48 is administered in 2 doses. PS48 is administered about 1 hour to about 96 hours prior to miR transfection and about 1 hour to about 96 hours after miR transfection, e.g., about 12 hours to about 72 hours or about 24 hours to about 60 hours prior to and after miR transfection. Preferably, PS48 is administered in two doses: one dose at 48 hours prior to miR transfection and one dose at 48 hours post-transfection.

A83-01(3-(6-Methyl-2-pyridinyl)-N-phenyl-4-(4-quinolinyl)-1H-pyrazole-1-c arbothioamide; C25H19N5S) is a TGFβ kinase/activin receptor like kinase (ALKS) inhibitor. It blocks the phosphorylation of Smad2 and inhibits TGFIβ-induced epithelial-to-mesenchymal transition. A83-01 is more potent than small molecule SB431542, and inhibits differentiation of rat induced pluripotent stem cells (iPSCs) and increases clonal expansion efficiency. Small molecule A83-01 helps maintain homogeneity and long-term in vitro self-renewal of human iPSCs. This molecule is soluble in DMSO to 100 mM. A83-01 is available from Stemgent, and used at a final concentration of about 0.01 μM to about 10 μM, e.g., about 0.1 μM to about 5 μM or about 0.4 μM to about 0.6 μM. Preferably, A83-01 is used at a final concentration of about 0.5 μM. A83-01 is administered in about one dose to about 5 doses, e.g., about 1 dose, about 2 doses, about 3 doses, about 4 doses, or about 5 doses. Preferably, A83-01 is administered in 2 doses. A83-01 is administered about 1 hour to about 96 hours prior to miR transfection and about 1 hour to about 96 hours after miR transfection, e.g., about 12 hours to about 72 hours or about 24 hours to about 60 hours prior to and after miR transfection. Preferably, A83-01 is administered in two doses: one dose at 48 hours prior to miR transfection and one dose at 48 hours post-transfection.

### III. Methods of the Invention

A. In Vivo Methods

The methods disclosed herein provide a solution to the clinical problem of non-functional scar tissue in an organ such as the heart following injury or disease. The methods lead to direct reprogramming of differentiated cells such as fibroblasts into cardiomyocytes or cardiomyocyte progenitors. Reprogramming is a process by which cells change phenotype, state of differentiation, or function. Accordingly, methods for promoting the conversion of a cell (such as a cardiofibroblast or dermal fibroblast) and/or cardiac fibrotic tissue into a cardiomyocyte and/or cardiomyocytic tissue are carried out by contacting fibrotic tissue (e.g., scar tissue) with any of the reprogramming media disclosed herein. Optionally, the cell and/or cardiac fibrotic tissue are also contacted with one or more microRNAs (such as any of those described above) and/or one or more reprogramming efficiency-enhancing molecules to enhance the efficiency of direct reprogramming Such molecules suitable for increasing the efficiency of conversion to cardiac myocytes include bone morphogenetic protein 4 (BMP4), Janus protein tyrosine kinase (JAK)-1 inhibitor [e.g., 2-(1,1-Dimethylethyl)-9-fluoro-3,6-dihydro-7H-benz[h]-imidaz[4,5-f]isoquinolin-7-one, Pyridone 6, P6, DBI (420099 JAK Inhibitor I)], RG108, R(+)Bay K 8644, PS48, A83-01, and histone deacetylase inhibitors (HDIs) such as valproic acid.

The fibrotic tissue to be treated is typically present in a heart diagnosed as having experienced cardiac myocardial infarction or other forms of cardiac disease, such as ischemic heart disease, hypertrophic cardiomyopathies, valvular heart disease, and/or congenital cardiomyopathies. For example, the tissue can be contacted with reprogramming media and, optionally, viral (e.g., lentiviral) constructs expressing microRNAs after fibrosis has developed as a result of myocardial infarction or other cardiac disease process, e.g., days (1, 2, 3, 4, 5, 6 days after), weeks (1, 2, 4, 6, 8), months (2, 4, 6, 8, 10, 12), or even a year or more after the primary tissue insult. The fibrotic tissue is contacted in situ.

In the case in which the organ is treated in a subject, e.g., a human patient, the compositions (i.e. the reprogramming media, the miRNAs, and/or a reprogramming efficiency-enhancing molecule) are delivered locally or systemically, e.g., using intravenous administration or direct injection into cardiac tissue. Other delivery schemes include oral, nasal, intradermal, transdermal, subcutaneous, intramuscular, intraperitoneal, suppository, and sublingual administration. For example, the compositions are administered by direct injection into cardiac tissue. Other delivery modes are characterized by sustained release, controlled release, or delayed release. Administration of the compositions may be via any common route so long as the target tissue is available via that route.

The compositions are administered as pharmaceutically acceptable compositions, e.g., formulated with a pharmaceutically acceptable carrier or excipient. For in vivo uses, the amounts and routes of administration will depend on numerous factors, including the amount of cardiofibroblast cells to be reprogrammed, severity of tissue damage, means of administration, and the like. In some embodiments, the reprogramming media is directly injected into the heart of the subject. In such embodiments, the amount needed may be about 1 mL, 2 mL, 3 mL, 4 mL, 5 mL mL to 10 mL, 20 mL, 30 mL, 40 mL, 50 mL, 60 mL, 70 mL, 80 mL, 90 mL, 100 mL or more, inclusive of values falling in between these numbers. In other embodiments, the reprogramming media may be administered via lavage (e.g., soaking the heart of the subject). In such embodiments, the amount of reprogramming media needed may be about 1 L, 2 L, 3 L, 4 L, 5 L or more, inclusive of values falling in between these numbers. In general, dosage is from 0.01 μg to 100 g per kg of body weight, from 0.1 μg to 10 g per kg of body weight, from 1.0 μg to 1 g per kg of body weight, from 10.0 μg to 100 mg per kg of body weight, from 100 μg to 10 mg per kg of body weight, or from 1 mg to 5 mg per kg of body weight, and may be given once or more daily, weekly, monthly or yearly. Examples of dosages based on small animal studies are in the range of 80 mg/kg for single or multiple dosages. However, it is expected with appropriate modification dosages 1-25 mg/kg for single to three repeated dosages will confer clinical benefit in human subjects.

B. In Vitro/Ex Vivo Methods

An alternative method for restoring tissue-specific function to fibrotic tissue in an organ is carried out by providing patient-derived cells (such as fibroblasts) and contacting them with any of the reprogramming media described herein. For example, the cells are dermal fibroblasts obtained from the skin of the patient to be treated. Alternatively, the cells are cardiac fibroblasts or epidermal keratinocytes. In other embodiments, the cells are derived from harvested scar tissue obtained from the heart of a subject who as recently experienced an adverse cardiac event such as, but not limited to, myocardial infarction or other forms of cardiac disease, such as ischemic heart disease, hypertrophic cardiomyopathies, valvular heart disease, and/or congenital cardiomyopathies. The cells can also be adiopocytes or CD34+ umbilical cord blood cells. A skin or other tissue biopsy is obtained from a patient using known methods. To extract fibroblasts, the tissue is minced in a buffer, e.g, PBS, into small fragments and cultured in cell culture media. Fibroblasts grow out of the minced pieces of tissue. Patient-specific induced pluripotent stem cells are made by performing a tissue, e.g., skin biopsy procedure on the patient; extracting human fibroblast cells from the skin biopsy tissue; and reprogramming patient-specific fibroblast cells into the pluripotent stem cell stage using ITS supplemented cell culture media or ITS+ascorbic acid supplemented cell culture media. Thus in addition to ITS-driven reprogramming, the method can also include transfecting the cells with a microRNA or combination of microRNAs (such as any of those described herein) known to cause direct reprogramming of cells into cardiomyocytes (such as, but not limited to, one or more of miR-1, miR-133, miR-208, miR-499). The transfection can occur ex vivo or in vitro.

Cells directly reprogrammed in this manner are useful for cell replacement therapy, in which the reprogrammed cells are infused or injected into an anatomical site that requires repair or regeneration of tissue. In some embodiments, the reprogrammed cells are delivered locally or systemically, e.g., using intravenous administration or direct injection into cardiac tissue. In other embodiments, the cells are delivered to the heart (e.g., to an area of the heart characterized by scar or fibrotic tissue) via surgery, catheter, or intra-cardiac injection (e.g., through the chest wall).

Accordingly, provided herein are methods for reprogramming a cell into a cardiomyocyte in a subject in need thereof comprising: (a) removing at least one cell from the subject; (b) contacting the at least one cell with a chemically defined reprogramming media in an amount and time sufficient to reprogram the cell into a cardiomyocyte, the media comprising advanced-DMEM/F12 media, bovine serum albumin, insulin-transferrin-selenium, L-glutamine, and ascorbic acid; and (c) administering the newly reprogrammed cardiomyocyte to the subject. Optionally, the method can also include transfecting the cells with a microRNA or combination of microRNAs described above prior to, coincident with, or subsequent to contacting them with the RM. The method can further include contacting the cells with any of the reprogramming efficiency-enhancing molecules discussed above. The cells contemplated for use in any of the in vitro/ex vivo methods described herein can be any cell capable of direct reprogramming into a cardiomyocyte. These include, but are not limited to, fibroblasts (such as cardiofibroblasts), adipocytes, or CD34+ umbilical cord blood cells.

The amount of media used in conjunction with the methods disclosed herein will depend on the specific method used. For in vitro/ex vivo uses, the amount of media needed is that sufficient to culture the cell (such as a cardiofibroblast) and will be dependent on factors such as the number of cells being cultured, the size of culturing flask used, and the like and can be determined by one skilled in the art. As an example, not intended to be limiting in any way, suitable volumes may range from 1 mL, 2 mL, 3 mL, 4 mL, 5 mL to 10 mL, 20 mL, 30 mL, 40 mL, 50 mL, 60 mL, 70 mL, 80 mL, 90 mL, 100 mL, or more, inclusive of values falling in between these numbers. Cells cultured in the RM media according to any of the methods disclosed herein can then be transferred to an injured region of the heart characterized by the presence of fibrotic tissue.

The amount of time needed to reprogram the cell will depend on the level of differentiation desired. Suitable culture times may range from at least 6, 12, 24, 48 hours, e.g., about 3-7 days. For example, the cells are cultured in RM for any of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, or 25 days or more, if desired. In some embodiments, the cells are cultured in the media for 3 days. In other embodiments, the cells are cultured in the media for 7 days. In other embodiments, the cells are cultured in the media for 14 days. In yet other embodiments, the cells are cultured in the media for 21 days.

C. Cardiomyocyte Marker Proteins

The level of differentiation of cells directly reprogramed into cardiomyocytes according to any of the methods disclosed herein can be determined by assessing the expression level of one or more cardiomyocyte marker proteins. Suitable cardiomyocyte marker proteins for use in the methods disclosed herein include, but are not limited to, Nanog, Oct3, Sox2, Klf4, Hand2, Tbx5, Mesp1, Mef2c, Tnni3, Actn2, Nkx.2.5, aMHC, Cacnalc, Sen5a. Contacting or culturing cells (such as fibroblasts) in any of the reprograming media disclosed herein results in the expression of one or more cardiomyocyte marker proteins that are not normally expressed in the cells originally contacted by the reprogramming media.

In one embodiment, culturing or contacting cells (such as fibroblasts) with any of the reprograming media disclosed herein results in any of about a 1-3, 2-4, 3-5, 4-6, 5-7, 6-8, 7-9, 8-10, 9-11, 10-12, 11-13, 12-14, 13-15, 1-5, 2-6, 3-8, 4-9, 5-10, 6-11, 7-12, 8-13, 9-14, 10-15. 11-16, 12-17, 13-18, 14-19, or 15-20 fold increase in the expression of one or more cardiomyocyte marker proteins, such as any of those discussed above, in comparison to cells that are not cultured in reprograming media. In other embodiments, culturing or contacting the cells with any of the reprograming media disclosed herein results in any of about a 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more fold increase in the expression of one or more cardiomyocyte marker proteins in comparison to cells that are not cultured in reprograming media.

In other embodiments, culturing or contacting cells (such as fibroblasts) that have been transfected with one or more miRNAs (such as any of the miRNAs disclosed herein) with any of the reprograming media disclosed herein results in any of about a 5-10, 7-12, 9-14, 10-16, 12-17, 13-18, 14-19, 15-20, 16-21, 17-22, 18-23, 19-24, 20-25, 25-35, 30-40, 35-45, 40-50, 45-55, 50-60, 55-65, 60-70, 65-75, 70-80, 75-85, 80-90, 85-95, 90-100, 95-105, 100-110, 105-115, 110-120, 115-125, 120-130, 125-135, 130-140, 135-145, 140-150, 145-155, 150-160, 155-165, 160-170, 165-175, 170-180, 175-185, 180-190, 185-195, 190-200, 195-205, 200-210, 205-215, 210-220, or 215-225 fold increase in the expression of one or more cardiomyocyte marker proteins, such as any of those discussed above, in comparison to cells that are not cultured in reprograming media. In further embodiments, culturing or contacting cells (such as fibroblasts) that have been transfected with one or more miRNAs (such as any of the miRNAs disclosed herein) with any of the reprograming media disclosed herein results in any of about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225 or more fold increase in the expression of one or more cardiomyocyte marker proteins, such as any of those discussed above, in comparison to cells that are not cultured in reprograming media.

D. Methods for Drug Testing

Also provided herein are methods for using the reprogrammed cardiomyocytes produced by any of the methods disclosed herein to test or screen for compounds capable of improving the ability of the heart to beat or to stop heart cells from beating (e.g. for cardioplegia). For these methods, one or more test compounds (such as, but not limited to, a small molecule, a therapeutic polypeptide or fragment thereof, an antibody or fragment thereof, an antisense nucleic acid, an siRNA, or a ribozyme) is contacted with the reprogrammed cardiomyocytes and the effect on cardiomyocyte beating is assessed. Alternatively, in other embodiments, the reprogrammed cardiomyocytes produced by any of the methods disclosed herein can be used to screen drugs intended for purposes other than those related to cardiology (e.g., kidney medication) to assure that a side effect does not include stopping/impairing the ability of cardiomyocytes to beat. The directly reprogrammed cardiomyocytes disclosed herein are also appropriate for use in direct screening assays or pharmacogenomics analysis.

### IV. Kits

The present disclosure further provides a kit for the reprogramming of cardiac fibroblasts into cardiomyocytes in a subject, the kit comprising, consisting of, or consisting essentially of, a chemically defined media which contains ITS or both ITS and ascorbic acid according to the present disclosure, a means of administering the media to a subject, the means including, but are not limited to, a syringe, a squirt bottle, a beaker, a flask, and the like, and instructions for using the kit components. In another example, the kit contains a liquid base media, a stock solution of ITS, and/or a stock solution of ascorbic acid along with instructions regarding preparation and use of a reprogramming media to effect conversion of a fibroblast to a cardiomyocyte. In some embodiments where the cells are to be treated ex vivo, the kit may further comprise cell culture equipment, such as cell culture flasks, petri dishes and the like, means of removing cardiac fibroblasts from a subject, such as a scalpel, syringe, or other biopsy-related tools, a means for readministering the reprogrammed cells to the patient, such as a syringe, and instructions for use.

### V. Medical Devices

Also provided herein are medical devices comprising a purified population of primary cells and any of the reprogramming media disclosed herein. The purified population of primary cells can also have been previously transfected with any of the microRNAs described herein capable of directly reprogramming cells (such as fibroblasts, for example, patient-derived fibroblasts) into cardiomyocytes. In some embodiments, said device is a stent or a catheter.

### EXAMPLES

The following examples are offered by way of illustration and not by way of limitation.

### Example 1: Reprogramming of Cardiac Fibroblasts to Cardiomyocytes by microRNAs is Augmented by Ascorbic Acid

This example demonstrates that RM augments miR direct reprogramming of fibroblasts into cardiomyocytes.

Isolation of Neonatal Tail-Tip and Cardiac Fibroblasts.

Mouse (C57BL/6) neonatal cardiac fibroblasts or tail tip fibroblasts were isolated from 2 day old mouse neonates according to known methods e.g., outlined in Jayawardena et al [Jayawardena T, Mirotsou M, Dzau V J. Direct reprogramming of cardiac fibroblasts to cardiomyocytes using microRNAs. Methods in molecular biology. 2014; 1150:263-272]. Cells were cultured in growth media containing DMEM (ATCC) supplemented with 15% v/v FBS and 1% v/v penicillin/streptomycin. Cells were used at passage 1 or passage 2.

Chemically Defined Reprogramming Media.

Cells were seeded at 5000 cells/cm² in growth media. Twenty-four hours later growth media was replaced with chemically defined reprogramming media (Advanced DMEM/F12, 0.2% w/v BSA, 250 μM Ascorbic Acid, 1× Insulin-Transferrin-Selenium, 1× L-Glutamine, 1× Penicillin-Streptomycin). Fresh media was added every two days.

MicroRNA transfection.

Fibroblasts were seeded into a 24 well-plate at 9,000 cells/well. After 24 hours, the cells were transfected with transfection reagent alone (Dharmafect-I, ThermoScientific), with transfection reagent plus non-targeting microRNAs (neg-miR), or with transfection reagent plus our previously reported combination of cardiac reprogramming microRNAs [Jayawardena T M, Egemnazarov B, Finch E A, et al. MicroRNA-mediated in vitro and in vivo direct reprogramming of cardiac fibroblasts to cardiomyocytes. Circulation research. 2012; 110:1465-1473]. (miR combo, miR-1, miR-133, miR-208, miR-499). The transfection method has been previously reported by Jayawardena et al [cited above].

qPCR.

Total RNA was extracted using Quick-RNA MiniPrep Kit according to the manufacturer's instructions (Zymo Research). Total RNA (50 ng-100 ng) was converted to cDNA using a high capacity cDNA reverse transcription kit (Applied Biosystems). cDNA was used in a standard qPCR reaction involving FAM conjugated gene specific primers and TaqMan Gene Expression Master Mix (Applied Biosystems). The following primers were used for qPCR: Gapdh (Mm99999915_m1), Nanog (Mm02384862_g1), Oct4/Pou5f1 (Mm03053917_g1), Rexo1 (Mm00617735_m1), Sox2 (Mm03053810_s1), Klf4 (Mm00516104_m1), Tnni3 (Mm00437164_m1), Actn2 (Mm00473657_m1), Cacnalc (Mm00437917_m1), Scn5a (Mm00451971_m1), Mesp1 (Mm00801883_g1), Nkx2-5 (Mm00657783_m1), Tbx5 (Mm00803518_m1), and Hand2 (Mm00439247_m1).

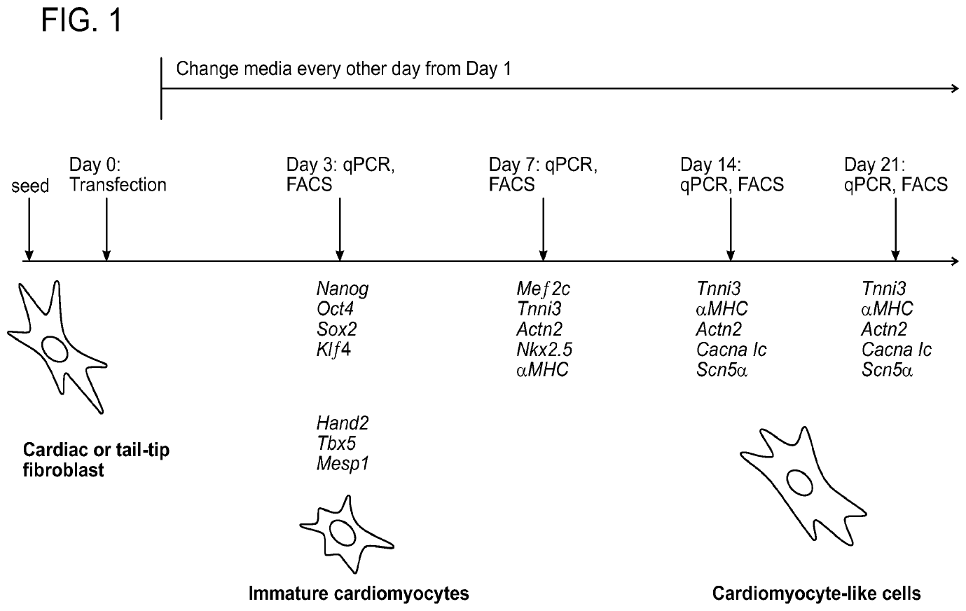

As shown in FIG. 1, neonatal cardiac or tail tip fibroblasts were isolated and cultured in fibroblast growth media (GM). The GM comprises the following: DMEM (ATCC#30-2002), 15% v/v embryonic stem cell qualified fetal bovine serum, and 1× penicillin/streptomycin. Following transfection with miR-combo or negative control miRs, fibroblasts were cultured in either regular growth media (GM) or chemically defined reprogramming media (RM). The RM comprises the following: Advanced-DMEM/F12 media, 0.2% bovine serum albumin, 1× insulin-transferrin-selenium, 1× L-glutamine, 250 μM ascorbic acid. Differentiation was assessed by qPCR, immunocytochemistry, and flow cytometry.

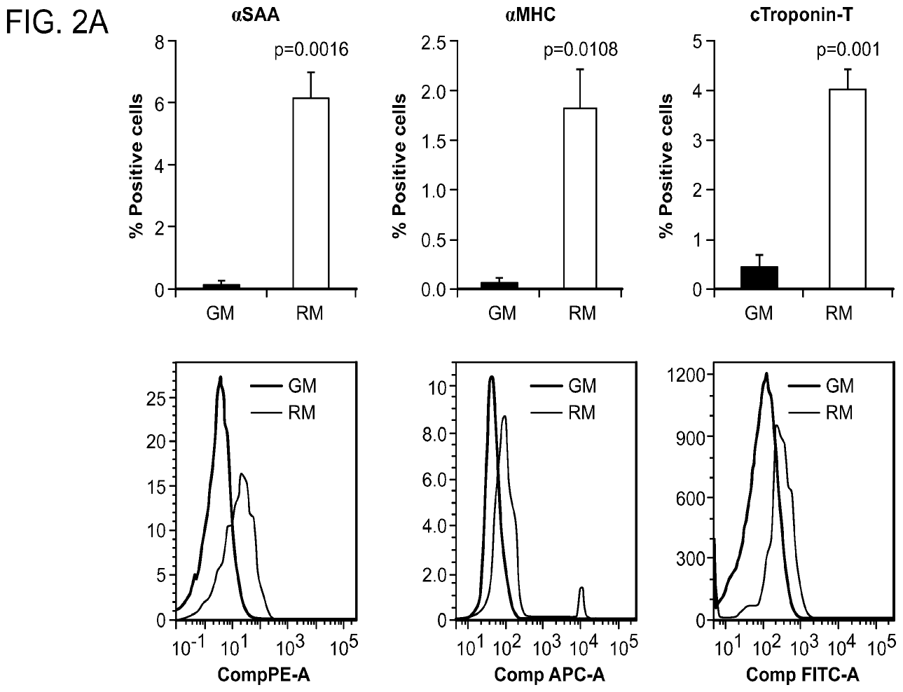

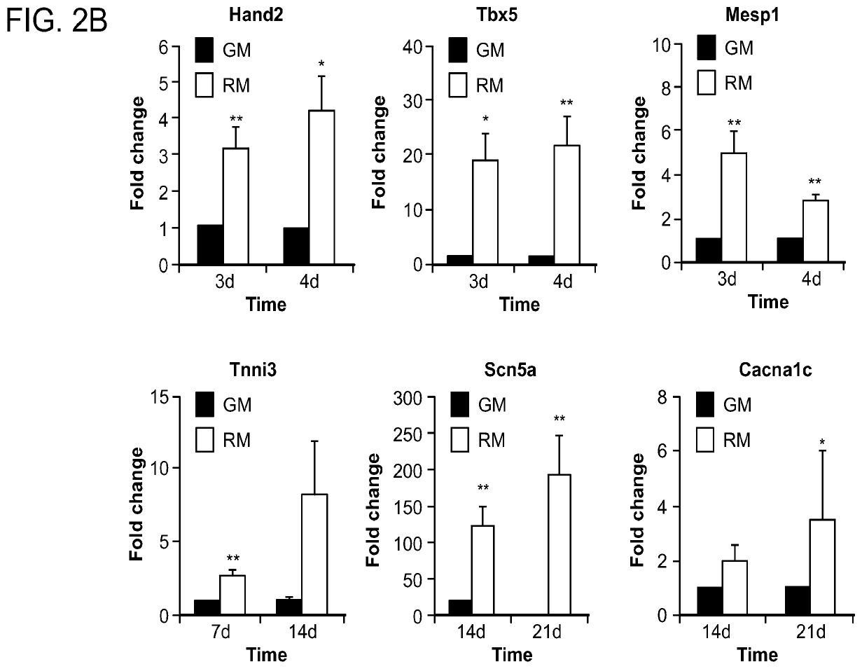

The addition of ascorbic acid to growth media augmented the effects of miR-combo upon cardiac gene expression (N=3, p<0.05). Compared to growth media, RM augmented miR-combo reprogramming of cardiac fibroblasts (N=4, p<0.01. Nkx2-5 5-fold in RM vs 1.5-fold in GM, Tnni3 15-fold vs 5-fold, Actn2 40-fold vs 5-fold, Myh6 15-fold vs 2-fold, Cacnalc 20-fold vs 5-fold, Scn5a 25-fold vs 2-fold) (FIGS. 2A-B).

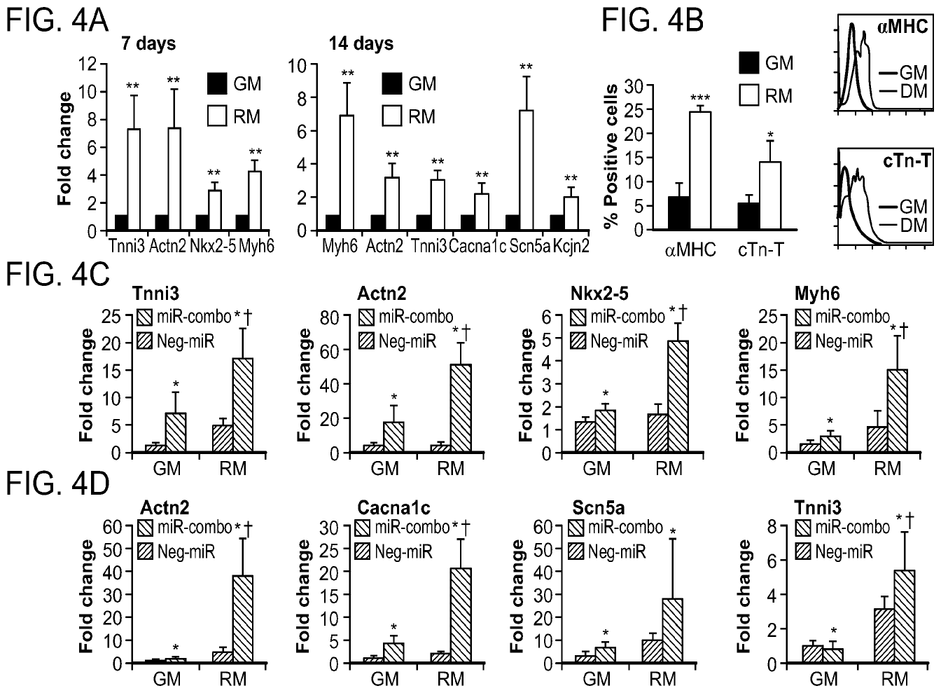

Increasing the concentration of ascorbic acid increased the additive effect on cardiac gene expression at both the gene and protein level (N=3, p<0.05). RM also increased expression of cardiac genes (N=3, p<0.05, Tnni3 3-fold, Scn5a 200-fold, Cacnalc 3-fold) and proteins (α-sarcomeric actinin, aMHC and cardiac troponin-T, N=3, 8 to 60-fold, p<0.01) in tail-tip fibroblasts (TTFs). GM had no effect on cardiac gene expression in TTFs (FIGS. 4A-D). As such, chemically defined media containing ascorbic acid as described herein augments microRNA directed cardiac reprogramming.

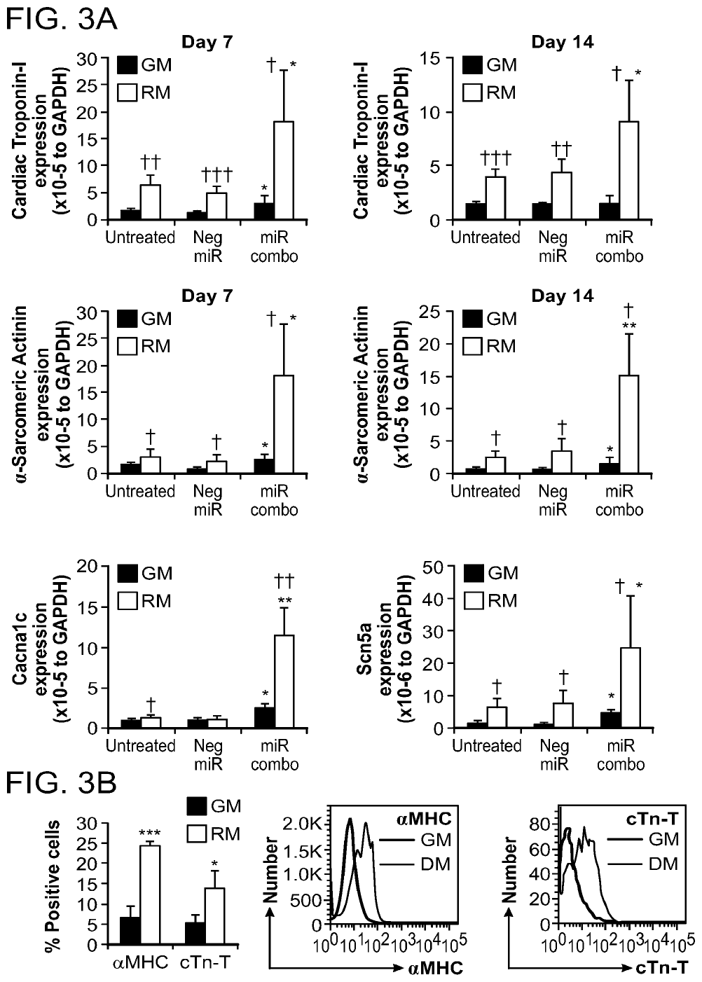

RM augmented the effect of miR combo for all of the cardiac markers tested with a fold increase of 2- to 20-fold (FIG. 3A). This effect was also observed in the absence of miR Combo. In control cells, (without microRNA) cardiac gene expression was similarly 2- to 10-fold higher in the reprogramming media treated group compared to the growth media treated group (FIG. 3A). Thus, reprogramming media was augmenting the effect of miR Combo by increasing baseline cardiac gene expression. Changes in gene expression are not necessarily reflected in protein levels so Cardiac Troponin-T and α-MHC protein expression was measured by flow cytometry. After 14 days of treatment, neonatal cardiac fibroblasts exposed to reprogramming media were found to have significantly higher levels of Cardiac Troponin-T and α-MHC protein when compared to the growth media treated group (FIG. 3B).

In summary, this example demonstrates that RM can not only increase the efficacy of miR-mediated direct reprogramming of fibroblasts to cardiomyocytes, RM alone significantly induces reprograming of fibroblasts to cardiomyocytes, e.g., in the absence of added microRNAs or ascorbic acid.

### Example 2: RM Induces Differentiation of Tail-Tip Fibroblasts into Cardiomyocytes

This example describes the effect of reprogramming media upon cardiac marker expression further. To eliminate the possibility that the results were due to the differentiation of cardiac progenitors within our cardiac fibroblast isolations, the experiments from Example 1 were repeated with neonatal tail-tip fibroblasts.

Neonatal tail-tip fibroblasts were isolated as described above and cultured in either fibroblast growth media (GM) or reprogramming media (RM). qPCR was performed as described above.

Flow Cytometry.

Cells were seeded in 6-well plate at 40,000 cells/well and fixed, when appropriate, with 4% v/v paraformaldehyde for 15 minutes at 4° C. Cells were washed with FACS buffer (1×PBS, 2 mM EDTA, 5% w/v BSA, 0.2% w/v saponin) at 800 g for 5 minutes at 4° C. Following washing cells were incubated in FACS buffer for 1 hour at 4° C. with the following antibodies (0.4 μg/10⁶ cells): αMHC (Abcam, ab15), αSarcomeric Actinin (Abcam, Ab68167), cardiac troponin-T (Abcam, FITC conjugate, ab105439), SSEA-1 (StemGent 09-0005), Oct4 (Abcam, ab18976). Cells were washed in FACS buffer. Where necessary, cells were incubated with anti-rabbit PE or anti-mouse APC conjugate in FACS buffer for 1 hour at 4° C. Cells were washed and re-suspended in FACS buffer. Cells were analyzed by FACS on a FACSCantoII (BD Biosciences). FlowJo version 10 was used to compensate and analyze the data.

Immunofluorescence.

Cells were fixed with 2% v/v paraformaldehyde (EMS) as described previously [Hodgkinson C P, Naidoo V, Patti K G, et al. Abi3bp is a multifunctional autocrine/paracrine factor that regulates mesenchymal stem cell biology. Stem Cells. 2013; 31:1669-1682]. Fixed cells were blocked in antibody buffer (1% w/v BSA, 0.3% v/v Triton X-100, in PBS) for 1 hr at room temperature and then incubated with primary antibodies overnight at 4° C. in antibody buffer. αMHC (Abcam, ab15, 1:200), cTnI (Abcam, ab47703, 1:400), Oct4 (Abcam, ab18976, 1:300), and Nanog (Abcam, ab14959, 1:100) were used at indicated concentrations. Alexa-Fluor conjugated secondary antibodies (Invitrogen) were used at 1:1000 dilution in antibody buffer for 1 hr at room temperature. Nuclei were stained by DAPI at 1 μg/ml for 15 minutes at room temperature in PBS.

iPS Cell Culture.

Cells were cultured in Knockout DMEM with 15% v/v fetal bovine serum, 1× Glutamax, 1× non-essential amino acids, 1% v/v penicillin/streptomycin, 0.0007% v/v β-mercarptoethanol, and 0.1 U/mL LIF.

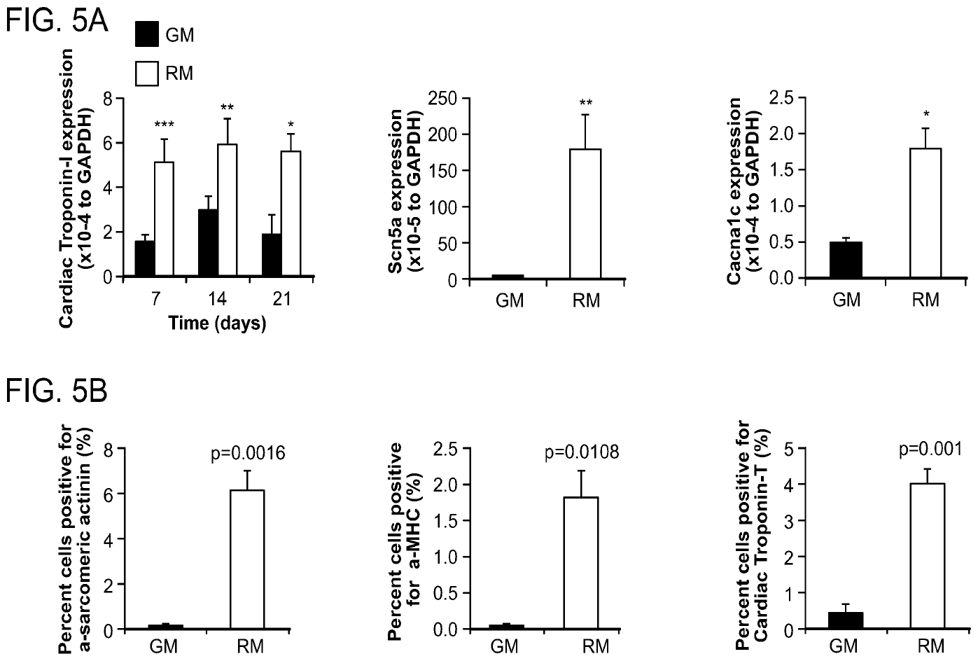

Reprogramming media significantly increased the mRNA levels of Cardiac Troponin-I, an intermediate cardiomyocyte marker, at days 7, 14 and 21 post-addition of media (FIG. 5A). Similarly, at day 21 post-addition of media, RM robustly stimulated expression of the mature cardiomyocyte markers Scn5a and Cacna1c (FIG. 5A). Studies were carried out to determine whether the changes in cardiac gene expression were reflected at the protein level. Neonatal tail-tip fibroblasts were cultured for 14 days in either growth media or reprogramming media. Cells were stained with antibodies for cardiac markers and then analyzed by flow cytometry. Reprogramming media increased the number of cells positive for α-sarcomeric actinin, α-myosin heavy chain, and cardiac troponin-T by greater than 5-fold, when compared to the growth media treated group (FIG. 5B).

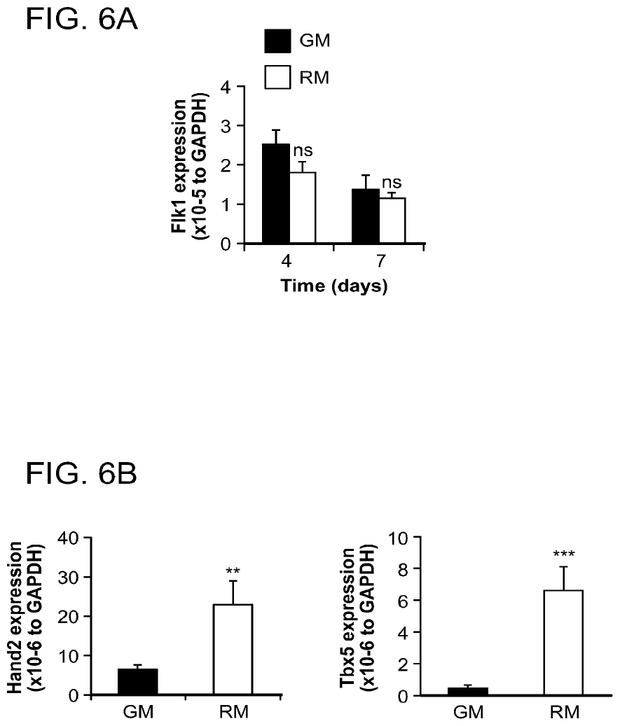

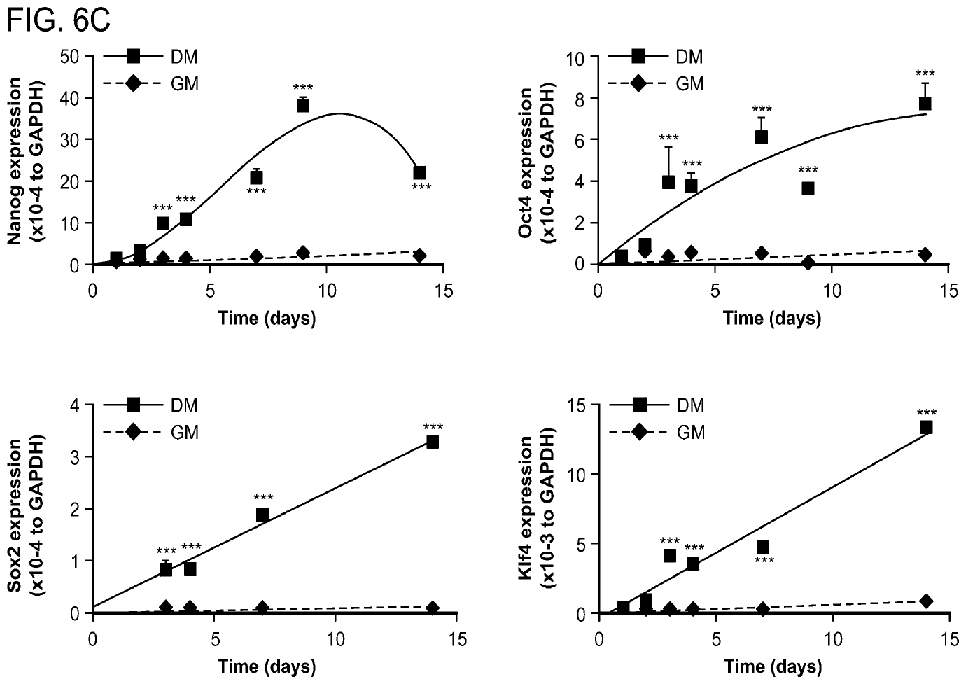

Experiments were carried out to determine the mechanism by which reprogramming media was inducing cardiac gene expression in neonatal fibroblasts. Flk-1 expression was not affected by reprogramming media suggesting that the neonatal tail-tip fibroblasts were not adopting a cardiac progenitor cell fate (FIG. 6A). Reprogramming media induced the expression of Hand2 and Tbx5, two early markers of commitment to the cardiac lineage, by 3- and 12-fold respectively (FIG. 6B). More dramatic effects were observed with pluripotency markers. Nanog mRNA levels in reprogramming media treated neonatal tail-tip fibroblasts reached >100-fold above that of cells cultured in growth media by day 7 (FIG. 6C).

| Human Nanog Nucleic Acid Sequence ACCESSION XM_01152085 | |
|---|---|
| (SEQ ID NO: 22) | |
| 1 | cacacccaca cgagatgggc acggagtagt cttgaaagac atgacaaatc accagacctg |
| 61 | ggaagaagct aaagagccag agggaaaaag ccagaagtcg actacctggg aggagggata |
| 121 | gacaagaaac caaactaaag gaaactaagt gtggatccag cttgtcccca aagcttgcct |
| 181 | tgctttgaag catccgactg taaagaatct tcacctatgc ctgtgatttg tgggcctgaa |
| 241 | gaaaactatc catccttgca aatgtcttct gctgagatgc ctcacacgga gactgtctct |
| 301 | cctcttcctt cctccatgga tctgcttatt caggacagcc ctgattcttc caccagtccc |
| 361 | aaaggcaaac aacccacttc tgcagagaag agtgtcgcaa aaaaggaaga caaggtcccg |
| 421 | gtcaagaaac agaagaccag aactgtgttc tcttccaccc agctgtgtgt actcaatgat |
| 481 | agatttcaga gacagaaata cctcagcctc cagcagatgc aagaactctc caacatcctg |
| 541 | aacctcagct acaaacaggt gaagacctgg ttccagaacc agagaatgaa atctaagagg |
| 601 | tggcagaaaa acaactggcc gaagaatagc aatggtgtga cgcagaaggc ctcagcacct |
| 661 | acctacccca gcctttactc ttcctaccac cagggatgcc tggtgaaccc gactgggaac |
| 721 | cttccaatgt ggagcaacca gacctggaac aattcaacct ggagcaacca gacccagaac |
| 781 | atccagtcct ggagcaacca ctcctggaac actcagacct ggtgcaccca atcctggaac |
| 841 | aatcaggcct ggaacagtcc cttctataac tgtggagagg aatctctgca gtcctgcatg |
| 901 | cagttccagc caaattctcc tgccagtgac ttggaggctg ccttggaagc tgctggggaa |
| 961 | ggccttaatg taatacagca gaccactagg tattttagta ctccacaaac catggattta |
| 1021 | ttcctaaact actccatgaa catgcaacct gaagacgtgt gaagatgagt gaaactgata |
| 1081 | ttactcaatt tcagtctgga cactggctga atccttcctc tcccctcctc ccatccctca |
| 1141 | taggattttt cttgtttgga aaccacgtgt tctggtttcc atgatgccca tccagtcaat |
| 1201 | ctcatggagg gtggagtatg gttggagcct aatcagcgag gtttcttttt tttttttttt |
| 1261 | cctattggat cttcctggag aaaa |
| Human Nanog Protein Sequence ACCESSION NP_001284627 | |
| (SEQ ID NO: 23) | |
| 1 | MSVDPACPQS LPCFEASDCK ESSPMPVICG PEENYPSLQM SSAEMPHTET VSPLPSSMDL |
| 61 | LIQDSPDSST SPKGKQPTSA EKSVAKKEDK VPVKKQKTRT VFSSTQLCVL NDRFQRQKYL |
| 121 | SLQQMQELSN ILNLSYKQVK TWFQNQRMKS KRWQKNNWPK NSNGVTQGCL VNPTGNLPMW |
| 181 | SNQTWNNSTW SNQTQNIQSW SNHSWNTQTW CTQSWNNQAW NSPFYNCGEE SLQSCMQFQP |
| 241 | NSPASDLEAA LEAAGEGLNV IQQTTRYFST PQTMDLFLNY SMNMQPEDV |

Reprogramming media induced Oct4 levels ˜40-fold, Sox2 levels by ˜30-fold, and Klf4 levels by ˜25-fold (FIG. 6C). Growth media had no effect on the expression of these pluripotency genes at any time point (FIG. 6C). Gene expression was verified experiments by flow cytometry. There was a significantly higher percentage of Oct4 and SSEA-1 positive cells in neonatal tail-tip fibroblasts cultured in reprogramming media when compared to the growth media treated group. The data indicated that reprogramming media induced neonatal tail-tip fibroblasts to become iPS cells. To investigate this further, neonatal tail-tip fibroblasts were cultured in growth and reprogramming media for 14 days and re-plated the cells onto a MEF feeder layer. These co-cultures were cultured in standard iPSC culture media to measure colony formation. Colony formation was observed with neonatal tail-tip fibroblasts cultured in reprogramming media but not growth media. 1-3 colonies were observed per 10,000 cells plated. Though these colonies tested positive for alkaline phosphatase, Nanog and Oct4 expansion was very limited indicating that they lacked the proliferative capacity of typical iPS cells.

### Example 3: Nanog Knockdown Significantly Inhibited Cardiac Gene Expression in Neonatal Tail-Tip Fibroblasts Cultured in Reprogramming Media

This Example tests whether Nanog is involved in the effect of reprogramming media upon cardiac gene expression.

Nanog siRNA Knockdown.

A Nanog siRNA pool (four siRNAs targeting Nanog) and a negative control were purchased from Dharmacon. siRNAs were made to 20□M in nuclease free water, aliquoted, and stored −80° C. until use. Fibroblasts were seeded into a 24 well-plate at 9,000 cells/well one day prior to transfection. On the day of transfection siRNAs were diluted to 5 μM in nuclease free water. For each well 5 μl of the working siRNA working solution was diluted with 95 μl Optimem-Serum Free In a separate tube 5 μl of dharmafect-I (Dharmacon) was diluted with 95 μl Optimem-Serum Free. After a 5 minute incubation the two solutions were combined. After 20 minutes complete media lacking antibiotics was added (800 μl) and the transfection complexes added to the cells.

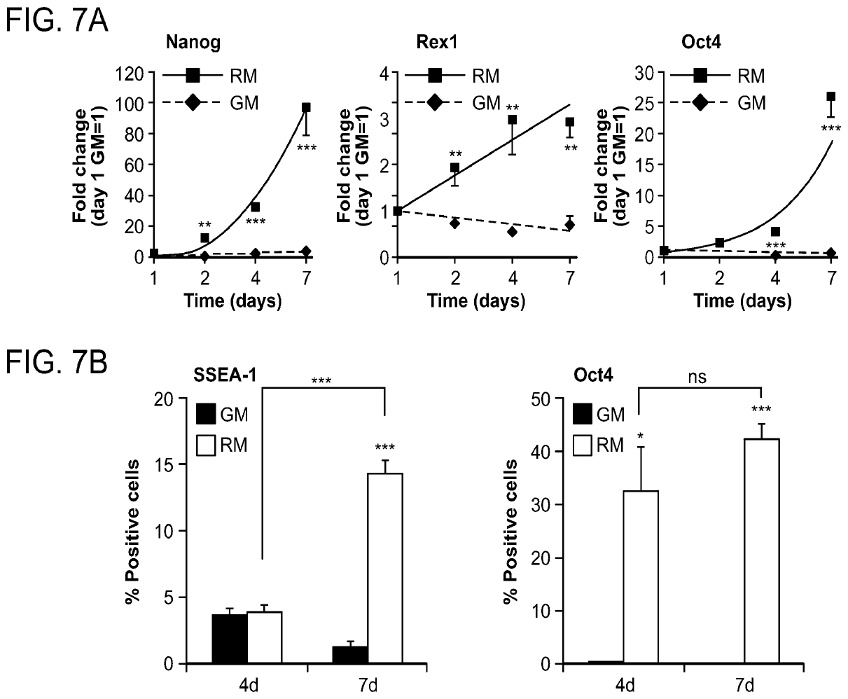

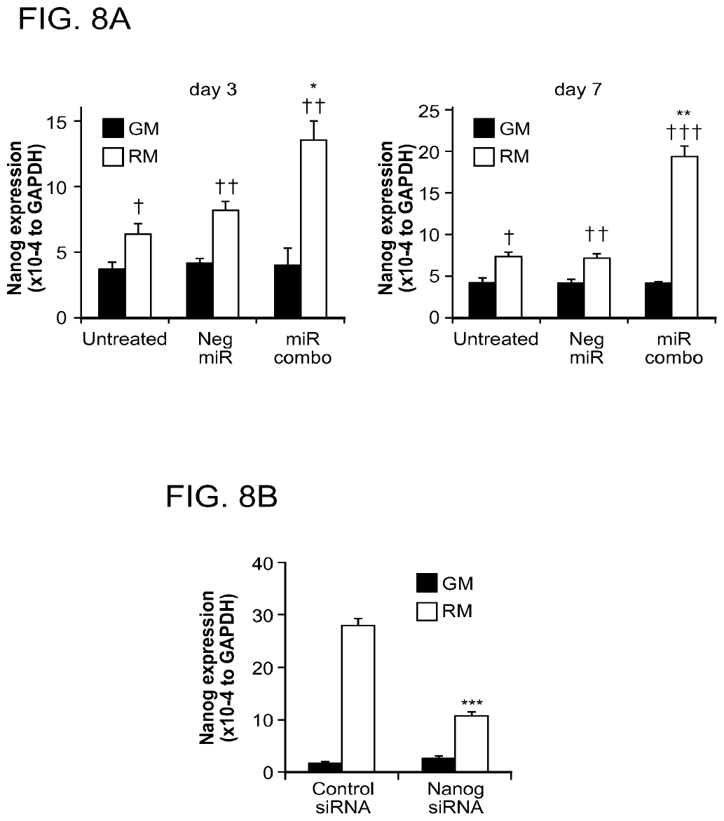

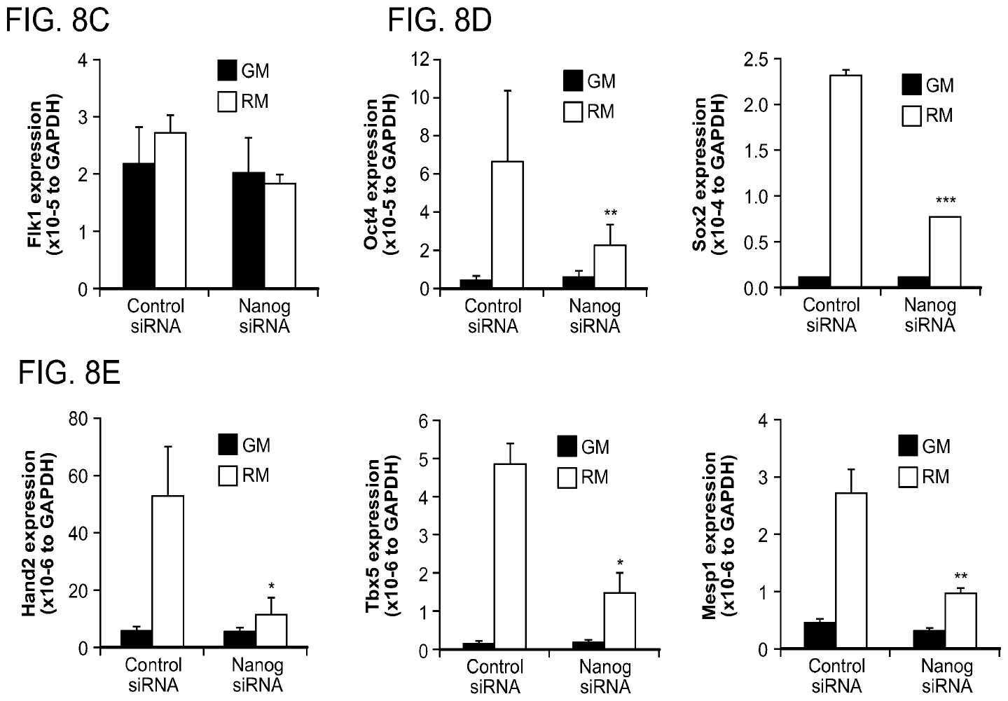

As described above, RM induced the expression of the pluripotency markers Nanog, Oct4 and Rex1 (N=5, p<0.01, Fold change >4-fold). GM had no effect on these markers. Nanog expression increased dramatically in neonatal tail-tip fibroblasts cultured in reprogramming media. Similarly, reprogramming media increased Nanog expression in neonatal cardiac fibroblasts (FIG. 8A). MiR combo had no effect on Nanog expression when cells were cultured in growth media. However, miR combo significantly augmented the effect of reprogramming media upon Nanog expression (FIG. 8A). Taking these results into consideration studies were carried out to determine if Nanog was responsible for the effect of reprogramming media upon cardiac gene expression. Neonatal tail-tip fibroblasts were transfected with either a negative control or Nanog siRNA. Nanog knockdown by siRNA completely inhibited the expression of cardiac markers (N=3, p<0.05) indicating that RM functions by inducing a pluripotent state (FIGS. 7A-B). Nanog knockdown was significant as determined by qPCR (FIG. 8B). Flk1 expression was not affected by Nanog siRNA (FIG. 8C). Knockdown of Nanog significantly inhibited the expression of the pluripotency markers Oct4 and Sox2 (FIG. 8D). Moreover, Nanog knockdown significantly inhibited cardiac gene expression in neonatal tail-tip fibroblasts cultured in reprogramming media for all the markers tested (FIG. 8E).

### Example 4: Determination of the Active Component of the Reprogramming Media

This Example demonstrates that insulin-transferrin-selenium is the active reprogramming component of RM.

Tail fibroblasts were isolated and cultured as described above. qPCR was performed as described above.

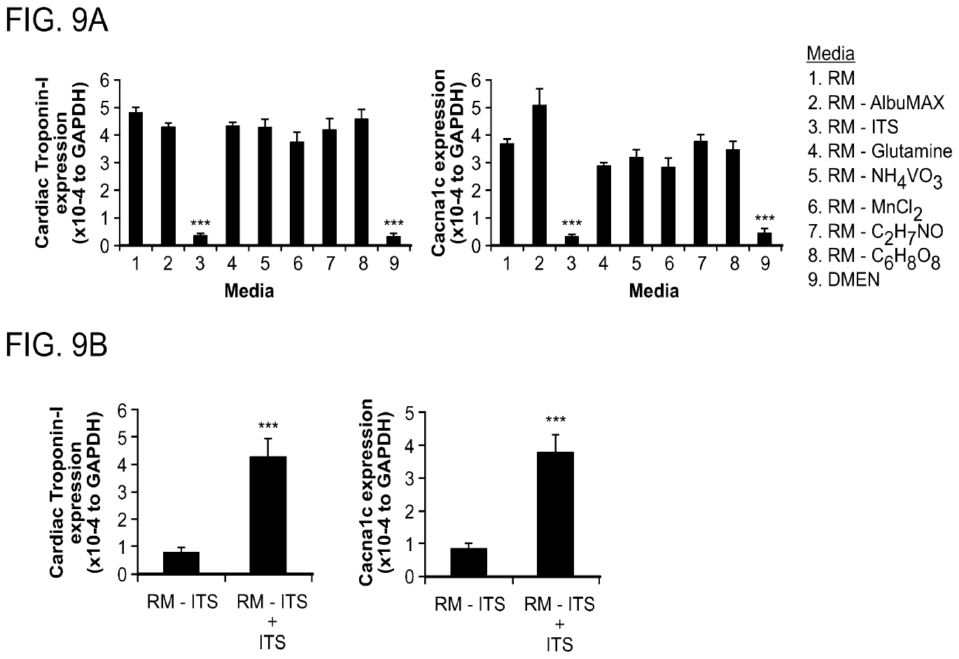

Studies were carried out to determine the active component of reprogramming media. One exemplary reprogramming media differs from standard DMEM by the inclusion of several specialized components: AlbuMAX, insulin-transferrin-selenium, ITS ascorbic acid (C6H806), glutathione, ammonium metavanadate (NH4VO3), Manganese chloride (MnCl2), and ethanolamine (C2H7NO) with ITS being the active component that promotes fibroblast-to-cardiomyocyte reprogramming Neonatal tail-tip fibroblasts were cultured in DMEM, reprogramming media, or reprogramming media lacking one of the specialized components. Cardiac gene expression was assessed after 14 days by qPCR. When compared to reprogramming media, DMEM had no effect upon Cardiac Troponin-I or Cacnalc expression (FIG. 9A). Only the removal of insulin-transferrin-selenium completely inhibited the effect of reprogramming media upon cardiac gene expression (FIG. 9A). Importantly the addition of insulin-transferrin-selenium to DMEM increased Cardiac Troponin-I and Cacnalc expression (FIG. 9B).

### Example 5: Characterization of the Cardiomyocyte-Like Cells

Reprogrammed cardiomyocytes harvested from the cardiac fibroblasts cultured in the chemically defined media in accordance with the present disclosure are assessed using known methods. For example, cardiomyocytes are subtyped, where expression of two major isoforms of MLC2 are used to identify atrial versus ventricular specification. Patch clamp analysis is used to evaluate the characteristics of action potentials.

Cells derived from culture in the chemically defined media containing ITS or ITS and ascorbic acid are administered (e.g., injected) into subjects after heart injury, e.g., myocardial infarction. The cells are administered days (e.g., 1-7 days), weeks (e.g., 1-4 weeks), months (e.g., 1-12 months) or even years after injury to a scarred site to reverse scarring and repair the myocardial tissue.

### OTHER EMBODIMENTS

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1.-7. (canceled)

8. A method of reprogramming a fibroblast cell comprising contacting the cell with a chemically defined reprogramming media comprising a base tissue culture media, insulin-transferrin-selenium or ascorbic acid in a somatic cell-reprogramming amount for a sufficient amount of time and volume such that the fibroblast is reprogrammed into a cardiomyocyte.

9. The method according to claim 8, the method further comprising transfecting into the cell at least one miRNA that is associated with facilitating the reprogramming of cells into cardiomyocytes prior to culturing in the chemically defined reprogramming media.

10. The method according to claim 9, wherein the miRNA is selected from the group consisting of miR-1, miR-133, miR-208, miR-499 and combinations thereof.

11. The method of claim 8, wherein the cell is selected from the group consisting of fibroblasts, adipocytes, or CD34+ umbilical cord blood cells.

12. The method of claim 11, wherein the fibroblast is a cardiofibroblast or dermal fibroblast.

13. The method of claim 8, wherein the cell comprises cardiac fibrotic tissue.

14. The method of claim 8, further comprising contacting the cell with a reprogramming efficiency-enhancing molecule.

15. The method of claim 14, wherein said molecule is one or more molecules selected from the group consisting of valproic acid, bone morphogenetic protein 4 (BMP4), JAK inhibitor 1, RG108, R(+)Bay K 8644, PS48, and A83-01.

16. A method of reprogramming a cell comprising a cardiofibroblast into a cardiomyocyte in a subject in need thereof comprising administering to the subject a therapeutically effective amount of a chemically defined reprogramming media such that the media contacts the cardiofibroblast for a sufficient amount of time that the cardiofibroblast is reprogrammed into a cardiomyocyte, the media comprising advanced-DMEM/F12 media, 0.2% bovine serum albumin, 1× insulin-transferrin-selenium, 1× L-glutamine, and 250 μM ascorbic acid.

17.-20. (canceled)

21. The method of claim 16, the method further comprising transfecting the cell with at least one miRNA capable of facilitating the reprogramming the cell into a cardiomyocyte prior to contacting the cell with the media.

22. The method according to claim 21, wherein the miRNA is selected from the group consisting of miR-1, miR-133, miR-208, miR-499 and combinations thereof.

23. The method of claim 16, further comprising contacting the cell with a reprogramming efficiency-enhancing molecule.

24. The method of claim 23, wherein said molecule is one or more molecules selected from the group consisting of valproic acid, bone morphogenetic protein 4 (BMP4), JAK inhibitor 1, RG108, R(+)Bay K 8644, PS48, and A83-01.

25. The method of claim 16, wherein said cardiomyocyte is characterized by an increased expression of a cardiomyocyte marker protein after said contacting step compared to the level of said marker protein before said contacting step.

26. The method of claim 25, wherein said marker protein is selected from the group consisting of Nanog, Oct3, Sox2, Klf4, Hand2, Tbx5, Mesp1, Mef2c, Tnni3, Actn2, Nkx.2.5, aMHC, Cacnalc, Sen5a.

27.-33. (canceled)
